(19) United States
(12) Patent Application Publication
(10) Pub. No.: US 2025/0161273 A1
(43) Pub. Date: May 22, 2025

(54) PHARMACEUTICAL COMPOSITION COMPRISING DIPHENYLDIAZOLE DERIVATIVES AND METHODS OF USE

(71) Applicant: MODAG GMBH, Wendelsheim (DE)

(72) Inventors: Armin GIESE, Wendelsheim (DE); Johannes LEVIN, Wendelshim (DE); Emanuela DEL VESCO, Verona (IT)

(73) Assignee: MODAG GMBH, Wendelsheim (DE)

(21) Appl. No.: 18/840,374

(22) PCT Filed: Mar. 1, 2023
(86) PCT No.: PCT/EP2023/055160

§ 371 Date: Aug. 21, 2024

(30) Foreign Application Priority Data
Mar. 1, 2022 (EP) 22159408.8

(51) Int. Cl. A61K 31/4155; A61K 9/48; A61P 25/16

(52) U.S. Cl. CPC A61K 31/4155; A61K 9/4858; A61P 25/16

(57) ABSTRACT

Provided herein are pharmaceutical compositions comprising a diphenyldiazole compound of general formula (A) or (A*) and methods of using the same for the treatment of neurodegenerative diseases, in particular α,-synucleinopathies.

### FIELD OF THE INVENTION

Provided herein are pharmaceutical compositions comprising at least one compound having the general formula (A) or (A*) and methods of using the same for the treatment of neurodegenerative diseases, in particular α-synucleinopathies.

### BACKGROUND

A large number of neurological and neurodegenerative diseases are known, many of which are presently not curable. Neurodegenerative diseases include Huntington's disease (HD), Hallervorden-Spatz disease, Alzheimer's disease (AD), senile dementia, Creutzfeldt-Jakob disease (CJD), arteriosclerotic dementia, Parkinson's disease (PD), cerebral thromboangiitis obliterans (Buerger's disease), and many others.

One class of neurodegenerative diseases, the α-synucleinopathies, are characterized by intracellular accumulation of protein aggregates, oligomers, protofibrils and fibrils, containing mainly α-synuclein. Examples of α-synucleinopathies are Parkinson's disease (PD), dementia with Lewy bodies (DLB) and multiple system atrophy (MSA).

The disease phenotype is dependent on the localization of pathological changes, which can predominantly affect the autonomic, nigro-striatal, ponto-cerebellar and cortical systems. MSA patients present dysautonomia combined with either predominant parkinsonism (MSA-P) or cerebellar ataxia (MSA-C) (Gilman, 2008). PD patients manifest predominantly with a hypokinetic-rigid phenotype (Berg, 2018), while patients with DLB show a mix of cognitive and motor disturbances (McKeith, 2017). No effective therapies to slow disease progression are available (Levin, 2016). Inhibition of α-synuclein aggregation is a rational therapeutic intervention targeting a key pathophysiological process of α-synucleinopathies (Wong, 2017; Meissner, 2019)

It is believed that the pathological effects on nerve cells in α-synucleinopathies are induced by the formation of small aggregates of α-synuclein (oligomers) and the subsequent formation of membrane pores. The α-synuclein oligomers have been shown to be the most relevant neurotoxic species and are targeted by compounds disclosed in International Patent Application No, PCT/EP2009/004144. One particular compound, anle138b, a small molecule compound shows strong disease-modifying effects in animal models of-synucleinopathies. In those studies, anle138b showed a high oral bioavailability and blood-brain barrier penetration leading to 5-fold higher levels of anle138b in the brain than in plasma (Wagner, 2013). In a range of different mouse models, anle138b strongly inhibited oligomer accumulation, neuronal degeneration, and disease progression in vivo (Wagner, 2013; Martinez Hernandez, 2018; Wagner, 2015; Brendel, 2019; Levin, 2014; Heras-Garvin, 2019; Wegrzynowicz, 2019).

Anle138b is a highly lipophilic molecule, which may explain its ability to cross the brain blood barrier and enter target cells, however it is a challenge for formulation development. An oral pharmaceutical composition useful for human administration, with a low fed/fasted effect, would be beneficial.

### SUMMARY OF THE INVENTION

Provided herein is a pharmaceutical composition comprising:
at least one compound having the general formula (A) or (A*)

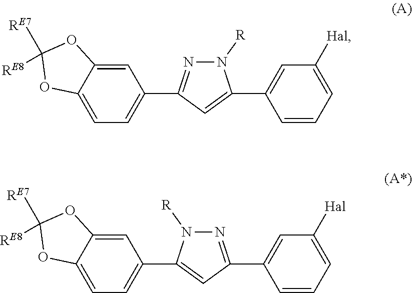

or a stereoisomer, racemate, hydrate or solvate thereof,
wherein
R is selected from hydrogen; C₁₋₄ alkyl; and —C₁₋₄ alkylene-halogen;
Hal is selected from F, Cl, Br, and I; and
R^{E7} and R^{E8} are independently H or F;
and a pharmaceutically acceptable excipient, wherein the excipient comprises at least one monoester of a fatty acid and polyethylene glycol and/or at least one diester of a fatty acid and polyethylene glycol,
wherein
the fatty acid is independently selected from C₈-C₂₂ fatty acids; and
the polyethylene glycol is independently selected from polyethylene glycols containing about 20 to about 40 ethylene oxide units. In some embodiments the PEG contains about 32 ethylene oxide units. In some embodiments, the fatty acid is independently selected from C₈-C₁₈ fatty acids.

In particular embodiments, the pharmaceutical composition comprises a compound wherein each of R, R^{E7} and R^{E8} is H and Hal is Br, the compound having the general formula (B), (B*)

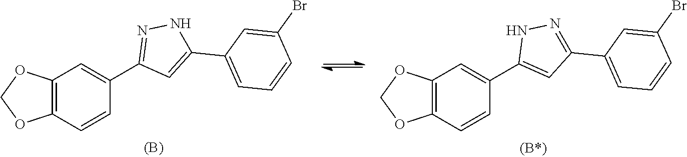

or a mixture thereof.
   The excipient can further comprise a monoglyceride of a fatty acid, a diglyceride of a fatty acid and/or a triglyceride of a fatty acid, wherein the fatty acid is independently selected from C₈-C₂₂ fatty acids. The excipient can further comprise a polyethylene glycol (PEG) containing about 20 to about 40 ethylene oxide units, and in specific embodiments about 32 ethylene oxide units. In some embodiments, the fatty acid is independently selected from C₈-C₁₈ fatty acids.

In some embodiments, the excipient comprises a mixture of monoesters of fatty acids and polyethylene glycol and/or diesters of fatty acids and polyethylene glycol, wherein the fatty acids maybe derived from a natural source, for example a plant source including coconut oil and/or hydrogenated coconut oil. In particular embodiments, the excipient comprises a mixture of monoesters of fatty acids and polyethylene glycol and/or diesters of fatty acids and polyethylene glycol, wherein the fatty acids comprise up to 15 wt % caprylic acid (C8), up to 12 wt % capric acid (C10), 30 to 50 wt % lauric acid (C12), 5 to 25 wt % myristic acid (C14), 4 to 25 wt % palmitic acid (C16), and 5 to 35 wt % stearic acid (C18).

In some embodiments, the excipient comprises about 50 wt % to about 80 wt %, preferably about 60 wt % to about 75 wt %, more preferably about 72 wt %, of the at least one monoester of a fatty acid and polyethylene glycol and/or the at least one diester of a fatty acid and polyethylene glycol. In some embodiments, the excipient further comprises about 10 wt % to about 30 wt %, preferably about 15 wt % to about 25 wt %, more preferably about 20 wt %, of the monoglyceride of the fatty acid, the diglyceride of the fatty acid and/or the triglyceride of the fatty acid. In some embodiments, the excipient further comprises about 5 wt % to about 20 wt %, preferably about 5 wt % to about 10 wt %, more preferably about 8 wt %, of the polyethylene glycol containing about 20 to about 40 ethylene oxide units, or about 32 ethylene oxide units.

The excipient can have a melting range in the range of about 33° C. to about 64° C., preferably about 35° C. to about 55° C., more preferably about 42.5° C. to about 47.5° C., even more preferably about 44° C. In some embodiments, the excipient has a hydrophilic lipophilic balance (HLB) of about 1 to about 16, preferably from about 7 to about 14, about 11 or about 14.

The process of obtaining the excipient is not limited. The excipient may be obtained by for example, an alcoholysis reaction between the polyethylene glycol and a triglyceride of the fatty acid or by polyglycolysis of hydrogenated vegetable oil with PEG, for example hydrogenated coconut oil or palm kernel oil with a PEG, for example PEG-32.

In some embodiments, the pharmaceutical composition comprises about 3 wt % to about 5 wt % of the compound having the general formula (A) or (A*) or a mixture thereof and about 95 wt % to about 97 wt % of the excipient, based on 100 wt % of the total pharmaceutical composition. In particular embodiments, the pharmaceutical composition comprises about 3 wt % to about 5 wt % of the compound having the general formula (B) or (B*) or a mixture thereof and about 95 wt % to about 97 wt % of the excipient, based on 100 wt % of the total pharmaceutical composition.

Further provided is an oral dosage form comprising the pharmaceutical described herein. The oral dosage form may be in the form of a capsule, for example a HPMC capsule or a gelatin capsule. In some embodiments the oral dosage form comprises from about 1 mg to about 100 mg of the compound, i.e. a compound of formulae (A), (A*), or mixtures thereof or (B), (B*), or mixtures thereof, from about 5 mg to about 50 mg of the compound, or from about 10 mg to about 30 mg of the compound.

Further provided herein are methods of treating or preventing a disease linked to protein aggregation and/or a neurodegenerative disease, for example an α-synucleinopathy, wherein a therapeutically effective amount of the pharmaceutical composition or oral dosage form disclosed herein is administered to a patient in need thereof. Further provided is the pharmaceutical composition or the oral dosage form for use in the treatment or prevention of a disease linked to protein aggregation and/or a neurodegenerative disease, for example, in the treatment or prevention of an α-synucleinopathy. In addition, provided is a compound selected from formulae (A), (A*), or mixtures thereof or (B), (B*), or mixtures thereof, and a pharmaceutically acceptable excipient comprising at least one monoester of a fatty acid and polyethylene glycol and/or at least one diester of a fatty acid and polyethylene glycol,
wherein the fatty acid is independently selected from C₈-C₂₂ fatty acids; and
the polyethylene glycol is independently selected from polyethylene glycols containing about 20 to about 40 ethylene oxide units,
   for the manufacture of a medicament for the treatment or prevention of a disease linked to protein aggregation and/or a neurodegenerative disease, for example, in the treatment or prevention of an α-synucleinopathy. The α-synucleinopathy can be selected from multiple system atrophy (MSA), Parkinson's disease (PD), or dementia with Lewy bodies (DLB), preferably multiple system atrophy (MSA). The pharmaceutical composition or medicament may be administered orally and is to be administered to a subject without regard to food intake.

Further provided are pharmaceutical compositions, unit dosage forms, and methods, according to any of the following clauses:

1. A pharmaceutical composition comprising a compound of formula (A), (A*) or a mixture thereof and an excipient comprising lauroyl polyoxyl-32 glycerides, the lauroyl polyoxyl-32glycerides comprising a mixture of mono-, di- and triglycerides, PEG fatty acid monoesters and/or diesters, and free PEG.
2. The pharmaceutical composition of clause 1, comprising a compound of formula (B), (B*) or a mixture thereof and an excipient comprising lauroyl polyoxyl-32 glycerides, the lauroyl polyoxyl-32 glycerides comprising a mixture of mono-, di- and triglycerides, PEG fatty acid monoesters and/or diesters, and free PEG.
3. The pharmaceutical composition of clause 2, comprising about 50 mg to about 300 mg of the compound of formula (B), (B*) or a mixture thereof.
4. An oral dosage form comprising the pharmaceutical composition of any one of clauses 1-3, particularly clause 2 or 3.
5. The oral dosage form of clause 4, wherein upon a single dose administration to a fasted healthy subject, the oral dosage form comprising 50 mg compound provides a geometric mean plasma Cmax of anle138b of about 54.3 ng/mL, the oral dosage form comprising 100 mg compound provides a geometric mean plasma Cmax of anle138b of about 156 ng/ml, the oral dosage form comprising 200 mg compound provides geometric mean plasma Cmax of anle138b of about 458 ng/ml or the oral dosage form comprising 300 mg compound provides geometric mean plasma Cmax of anle138b of about 704 ng/mL.
6. The oral dosage form of clause 4 or clause 5, wherein upon a single dose administration to a fasted healthy subject, the oral dosage form comprising 50 mg compound provides a geometric mean plasma AUC(0-24) of anle138b of about 113 ng*h/mL, the oral dosage form comprising 100 mg compound provides a geometric mean plasma AUC(0-24) of anle138b of about 366 ng*h/mL, the oral dosage form comprising 200 mg compound provides geometric mean plasma AUC(0-24) of anle138b of about 1090 ng*h/ml or the oral dosage form comprising 300 mg compound provides geometric mean plasma AUC(0-24) of anle138b of about 1650 ng*h/mL.
7. The oral dosage form of any one of clauses 4-6, wherein upon a single dose administration to a fasted healthy subject, the oral dosage form comprising 50 mg compound provides a geometric mean plasma T½ of anle138b of about 3.94 hr, the composition comprising 100 mg oral dosage form provides a geometric mean plasma T½ of anle138b of about 10.79 hr, the oral dosage form comprising 200 mg compound provides geometric mean plasma T½ of anle138b of about 12.76 hr or the oral dosage form comprising 300 mg compound provides geometric mean plasma T½ of anle138b of about 16.22 hr.
8. The oral dosage form of clause 4, wherein upon administration to a fasted healthy subject once daily for at least 7 days, the oral dosage form comprising 100 mg compound provides a geometric mean plasma Cmax of anle138b of about 135 ng/ml on day 1 and about 70.9 ng/ml on day 7, the oral dosage form comprising 200 mg compound provides a geometric mean plasma Cmax of anle138b of about 447 ng/ml on day 1 and about 128 ng/ml on day 7, or the oral dosage form comprising 300 mg compound provides a geometric mean plasma Cmax of anle138b of about 910 ng/ml on day 1 and about 307 ng/ml on day 7.
9. The oral dosage form of clause 4 or clause 8, wherein upon administration to a fasted healthy subject once daily for at least 7 days, the oral dosage form comprising 100 mg compound provides a geometric mean plasma AUC(0-tau) of anle138b of about 261 ng*h/mL on day 1 and about 141 ng*h/mL on day 7, the oral dosage form comprising 200 mg compound provides a geometric mean plasma AUC(0-tau) of anle138b of about 905 ng*h/mL on day 1 and about 308 ng*h/mL on day 7, or the oral dosage form comprising 300 mg compound provides a geometric mean plasma AUC(0-tau) of anle138b of about 2210 ng*h/mL on day 1 and about 633 ng*h/mL on day 7.
10. The oral dosage form of any one of clause 4, or clauses 8-9, wherein upon administration to a fasted healthy subject once daily for at least 7 days, the oral dosage form comprising 100 mg compound provides a geometric mean plasma T½ of anle138b of about 4.23 hr on day 7, the oral dosage form comprising 200 mg compound provides a geometric mean plasma T½ of anle138b of about 9.48 hr on day 7, or the oral dosage form comprising 300 mg compound provides a geometric mean plasma T½ of anle138b of about 6.07 hr on day 7.
11. The oral dosage form of clause 4, wherein upon a single dose administration to a healthy subject under fasted conditions, the oral dosage form comprising 150 mg compound provides a geometric mean plasma Cmax of anle138b of about 442 ng/ml and/or a geometric mean plasma AUC(0-24) of anle138b of about 896 ng*h/mL and/or a geometric mean plasma T½ of anle138b of about 11.3 hr.
12. The oral dosage form of clause 4, wherein upon a single dose administration to a healthy subject under fed conditions, the oral dosage form comprising 150 mg compound provides a geometric mean plasma Cmax of anle138b of about 196 ng/ml and/or a geometric mean plasma AUC(0-24) of anle138b of about 641 ng*h/mL and/or a geometric mean plasma T½ of anle138b of about 15.22 hr.
13. The oral dosage form of clause 4, wherein upon administration of the composition comprising 150 mg compound to a patient under fed conditions once daily for 7 consecutive days, provides on day 1 a geometric mean plasma Cmax of anle138b of about 462 ng/ml, and/or a AUC(0-24) of anle138b of about 1040 ng*h/mL.
14. The oral dosage form of clause 4, wherein upon administration of the oral dosage form comprising 150 mg compound to a patient under fed conditions once daily for 7 consecutive days, provides on day 7 a geometric mean plasma Cmax of anle138b of about 160 ng/ml, and/or a AUC(0-24) of anle138b of about 388 ng*h/mL and/or T½ of anle138b of about 11.7 h.
15. The oral dosage form of clause 4, wherein upon administration of the composition comprising 300 mg compound to a patient under fed conditions once daily for 7 consecutive days, provides on day 1 a geometric mean plasma Cmax of anle138b of about 933 ng/ml, and/or a AUC(0-24) of anle138b of about 2500 ng*h/mL.
16. The oral dosage form of clause 4, wherein upon administration of the composition comprising 300 mg compound to a patient under fed conditions once daily for 7 consecutive days, provides on day 7 a geometric mean plasma Cmax of anle138b of about 365 ng/ml, and/or a AUC(0-24) of anle138b of about 722 ng*h/mL and/or T½ of anle138b of about 12.1 h.
17. The oral dosage form of clause 4, wherein upon administration of the composition comprising 150 mg compound to a patient under fasted conditions twice daily for 7 consecutive days, provides on day 1 a geometric mean plasma Cmax of anle138b of about 546 ng/ml, and/or a AUC(0-24) of anle138b of about 993 ng*h/mL.
18. The oral dosage form of clause 4, wherein upon administration of the composition comprising 150 mg compound to a patient under fasted conditions twice daily for 7 consecutive days, provides on day 7 a geometric mean plasma Cmax of anle138b of about 182 ng/ml, and/or a AUC(0-24) of anle138b of about 297 ng*h/mL and/or T½ of anle138b of about 14.2 hr.
19. The pharmaceutical composition of any one of clauses 1-3 or the oral dosage form of any one of clauses 4-18, for use in the treatment or prevention of a disease linked to protein aggregation and/or a neurodegenerative disease.
20. The pharmaceutical composition for use or the oral dosage form for use of clause 19 wherein the disease is an α-synucleinopathy, for example wherein the synucleinopathy is multiple system atrophy (MSA), Parkinson's disease (PD), or dementia with Lewy bodies (DLB), preferably multiple system atrophy (MSA).
21. A method of treating or preventing a disease linked to protein aggregation and/or a neurodegenerative disease, the method comprising administering to a subject in need thereof a therapeutically effective amount of the pharmaceutical composition of any one of clauses 1-3 or the oral dosage form of any one of clauses 4-18.
22. The method of clause 21, wherein the disease is an α-synucleinopathy, for example wherein the synucleinopathy is multiple system atrophy (MSA), Parkinson's disease (PD), or dementia with Lewy bodies (DLB), preferably multiple system atrophy (MSA).

The oral dosage form may be administered in one or more units, wherein each unit comprises about 10 mg to about 50 mg, or about 10 mg, or 30 mg of the compound of formula (B), (B*) or a mixture thereof.

### FIGURES

FIG. 1 is a graph showing particle size distribution of nanomilled anle138b. The Y axis is volume (%), the x axis is particle size (μm) on a log scale.

FIGS. 2A-2D are graphs showing PK Cmax profiles of each of the 3 rats in each group in the rat formulation study, described in example 1.2.

FIG. 3 is a logarithmic plot of plasma concentrations of anle138b following single dose of DP in healthy volunteers.

FIGS. 4A and 4B are logarithmic plots of multiple dose plasma concentrations of anle138b DP on Day 1 (4A) and Day 7 (4B) in healthy volunteers.

FIG. 5 is a logarithmic plot of food effect on single dose plasma concentrations of anle138b DP.

### DETAILED DESCRIPTION OF THE INVENTION

The present disclosure relates to a pharmaceutical composition comprising at least one compound having the general formula (A) or (A*),
and a pharmaceutically acceptable excipient, wherein the excipient comprises at least one monoester of a fatty acid and polyethylene glycol and/or at least one diester of a fatty acid and polyethylene glycol.

The compositions according to the present disclosure have advantageous properties including safety, good bioavailability and/or little or no effect of food intake on bioavailability, specifically as measured by AUC.

The pharmaceutical compositions disclosed herein include, as an active pharmaceutical ingredient (API), bicyclic compounds that were disclosed in PCT/EP2009/004144 having the general formula (A) or (A*)

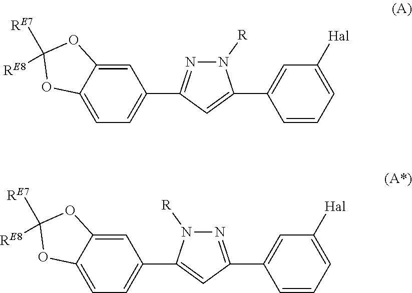

or a stereoisomer, racemate, hydrate or solvate thereof,
wherein
R is selected from hydrogen, C₁₋₄ alkyl, or C₁₋₄ alkylene-halogen;
Hal is F, Cl, I or Br; and
each of R^{E7} and R^{E8} is independently H or F.

In some embodiments, each of R^{E7}, R^{E8} and R is hydrogen (H).

In some embodiments, Hal is Br.

In some embodiments, the API is anle138b. As used herein “anle138b” is a diphenyl-pyrazole subspecies of general formulae (A) and (A*). It's chemical name is 3-(1,3-benzodioxol-5-yl)-5-(3-bromophenyl)-1H-pyrazole and it exists as tautomers having the following formulae (B) and (B*)

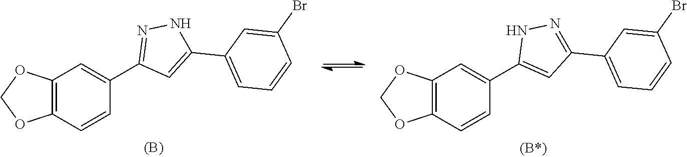

Anle138b represents a first-in-class compound that modulates toxic oligomers based on high-affinity binding to a structural epitope related to misfolding along the amyloidogenic pathway. It has been shown that binding destabilizes toxic oligomers, prevents the formation of oligomer pores in membranes and blocks prion-like propagation of α-synuclein aggregation (Wagner, 2013; Martinez Hernandez, 2018; Camilleri, 2020; Ghio, 2019). Anle138b showed structure-dependent binding to pathological aggregates and strongly inhibited formation of pathological oligomers in vitro and in vivo for α-synuclein as well as for other disease-relevant amyloidogenic proteins such as prion protein, Abeta (Amyloid beta, Aβ), and tau (Wagner, 2013; Martinez Hernandez, 2018; Camilleri, 2020; Ghio, 2019; Deeg, 2015; Reiner, 2018; Wagner, 2015). The molecular mode of action of anle138b was also studied by means of all-atom molecular dynamics simulations on the multi-microsecond time scale. This work provides insight into the binding mechanism of anle138b. anle138b was shown to bind to small oligomers and its ability to directly modulate these structures during the process of peptide aggregate formation. Importantly, anle138b does not bind to the monomer and therefore does not interfere with its physiological function. Without wishing to be bound to theory, anle138b was shown to reduce the overall number of intermolecular hydrogen bonds in oligomers, disfavor the sampling of the aggregated state, and remodel the conformational distributions within the small oligomer peptide aggregates (Matthes, 2017).

In principle, at least one compound having the general formula (A) or (A*) or any stereoisomer, racemate, hydrate, or solvate thereof may be used in the preparation of the pharmaceutical compositions disclosed herein.

It is understood that any tautomer, crystalline form or amorphous form of the at least one compound having the general formula (A) or (A*) may be used in the preparation of the pharmaceutical compositions disclosed herein.

In a preferred embodiment, anle138b (formulae (B), (B*) or mixtures) is utilized in the preparation of the pharmaceutical compositions disclosed herein.

In accordance with the present invention, the terms “formulation” and “pharmaceutical composition” may be used interchangeably and relate to a composition for administration to a subject, preferably a human patient. By “pharmaceutically acceptable excipient” is meant a non-toxic solid, semisolid or liquid carrier or diluent. In some embodiments the excipient imparts on the composition enhanced bioavailability relative to the unformulated compound.

The pharmaceutical compositions disclosed herein comprise the at least one compound having the general formula (A), (A*) or a mixture thereof and a pharmaceutically acceptable excipient, wherein the excipient comprises at least one monoester of a fatty acid and polyethylene glycol (PEG) and/or at least one diester of a fatty acid and polyethylene glycol (PEG). In addition, the excipient can further comprise a glyceride such as a monoglyceride of a fatty acid, a diglyceride of a fatty acid, a triglyceride of a fatty acid or a mixture thereof. Furthermore, the excipient can further comprise polyethylene glycol (PEG) which is not reacted with a fatty acid (i.e., “free polyethylene glycol” or “free PEG”).

In one embodiment, the excipient comprises a polyethylene glycol (PEG) fatty acid ester (particularly at least one monoester of a fatty acid and polyethylene glycol and at least one diester of a fatty acid and polyethylene glycol), a glyceride (particularly a monoglyceride of a fatty acid, a diglyceride of a fatty acid, and a triglyceride of a fatty acid) and free polyethylene glycol.

The polyethylene glycol and the fatty acid which are present in the monoester and/or diester of a fatty acid and polyethylene glycol, the glyceride and free polyethylene glycol, respectively, can be the same or different. For ease of manufacturing, the polyethylene glycol and the fatty acid, which are present in the PEG fatty acid ester, the glyceride and free polyethylene glycol, respectively, are the same.

The amounts of the at least one monoester and/or diester of a fatty acid and PEG in the excipient are not particularly limited and preferably range from about 50 wt % to about 80 wt %, more preferably about 60 wt % to about 75 wt %, even more preferably about 72 wt %. In a preferred embodiment, the excipient comprises about 10 wt % to about 30 wt %, more preferably about 15 wt % to about 25 wt %, even more preferably about 20 wt %, of the glycerides. Preferably, the excipient comprises about 5 wt % to about 20 wt %, more preferably about 5 wt % to about 10 wt %, even more preferably about 8 wt %, of the free polyethylene glycol, e.g. PEG 1500. In some embodiments, the excipient comprises about 72 wt % of at least one monoester and/or diester of a fatty acid and PEG, about 20 wt % of the glyceride and about 8 wt % free PEG.

All of the at least one monoester and/or diester of fatty acid and PEG, the glyceride and the free polyethylene glycol in the pharmaceutical composition of the present invention are considered part of the excipient and are taken into account when the above amounts are determined.

A fatty acid is an monocarboxylic acid having an aliphatic chain. The aliphatic chain can be branched or linear and is typically linear. In one embodiment, the aliphatic chain may include a hydroxy group substituent. A single type or a mixture of fatty acids can be employed as the fatty acid in the present invention. Due to the ready availability, mixtures of fatty acids can be chosen.

The fatty acid may be unsaturated (i.e. containing carbon-carbon double bonds) or saturated, typically saturated. If a mixture of fatty acids is used, the fatty acids will preferably be predominantly saturated, e.g., the mixture will contain at least 75 wt % saturated fatty acids, more preferably at least 80 wt % saturated fatty acids.

The composition of a fatty acid is identified, inter alia, by the length of its aliphatic chain. Fatty acids typically have aliphatic chains which are 8 to 22 carbons (C₈-C₂₂) in length, and in certain embodiments 8 to 18 carbons (C₈-C₁₈) in length. Examples of suitable fatty acids include caprylic acid (C8:0), capric acid (C10:0), lauric acid (C12:0), myristic acid (C14:0), palmitic acid (C16:0), stearic acid (C18:0), oleic acid (C18:1), linoleic acid (C18:2), γ-linoleic acid (C18:3), ricinoleic acid (C18:1, (OH)), arachidic acid (C20:0), and/or behenic acid (C22:0). Preferably the fatty acid comprises lauric acid (C12:0). More preferably the fatty acids comprise lauric acid (C12:0) in combination with myristic acid (C14:0), palmitic acid (C16:0), and/or stearic acid (C18:0) and optionally in combination with caprylic acid (C8:0) and/or capric acid (C10:0). Even more preferably the fatty acids include lauric acid (C12:0), myristic acid (C14:0), palmitic acid (C16:0), and stearic acid (C18:0) and optionally caprylic acid (C8:0) and/or capric acid (C10:0). Further preferably the fatty acids include caprylic acid (C8:0), capric acid (C10:0), lauric acid (C12:0), myristic acid (C14:0), palmitic acid (C16:0), and stearic acid (C18:0). (Cx:y) is a notation used for fatty acids wherein x refers to the number of carbon atoms in the fatty acid chain and y refers to the number of unsaturated carbon bonds in the fatty acid chain.

In the following, the weight percentages are based on the total weight of fatty acids.

Caprylic acid can be absent. In a preferred embodiment, the fatty acid includes caprylic acid, preferably in an amount of at least 0.5 wt %, more preferably at least 1 wt %, even more preferably at least 3 wt %. Preferably the fatty acid includes at most 20 wt %, more preferably at most 15 wt %, even more preferably at most 10 wt % caprylic acid. The fatty acid preferably comprises at most 15 wt % caprylic acid.

Capric acid can be absent. In a preferred embodiment, the fatty acid includes capric acid, preferably in an amount of at least 0.5 wt %, more preferably at least 1 wt %, even more preferably at least 3 wt %. Preferably the fatty acid includes at most 20 wt %, more preferably at most 15 wt %, even more preferably at most 10 wt % capric acid. The fatty acid preferably comprises at most 12 wt % capric acid.

In a preferred embodiment, the fatty acid includes lauric acid, preferably in an amount of at least 20 wt %, more preferably at least 30 wt %, even more preferably at least 40 wt %. Preferably the fatty acid includes at most 60 wt % lauric acid, more preferably at most 50 wt % lauric acid. The fatty acid preferably comprises 30 to 50 wt % lauric acid.

In a preferred embodiment, the fatty acid includes myristic acid, preferably in an amount at least 1 wt %, more preferably at least 5 wt %, even more preferably at least 10 wt %. Preferably the fatty acid includes at most 40 wt %, more preferably at most 30 wt %, even more preferably at most 25 wt % myristic acid. The fatty acid preferably comprises 5 to 25 wt % myristic acid.

In a preferred embodiment, the fatty acid includes palmitic acid, preferably in an amount of at least 1 wt %, more preferably at least 4 wt %, even more preferably at least 5 wt %, further preferably at least 10 wt %. Preferably the fatty acid includes at most 40 wt %, more preferably at most 30 wt %, even more preferably at most 25 wt % palmitic acid. The fatty acid preferably comprises 4 to 25 wt % palmitic acid.

In a preferred embodiment, the fatty acid includes stearic acid, preferably in an amount of at least 1 wt %, more preferably at least 5 wt %, even more preferably at least 10 wt %. Preferably the fatty acid includes at most 40 wt %, more preferably at most 35 wt %, even more preferably at most 30 wt %, further preferably at most 25 wt % stearic acid. The fatty acid preferably comprises 5 to 35 wt % stearic acid.

Mixtures of one or more of the above-mentioned fatty acids are also envisaged.

A mixture of fatty acids in a typical composition can be exemplified (in weight percent, wt %, based on the total weight of fatty acids) as follows:
up to 15 wt % caprylic acid,
up to 12 wt % capric acid,
30 to 50 wt % lauric acid,
5 to 25 wt % myristic acid,
4 to 25 wt % palmitic acid, and
5 to 35 wt % stearic acid.

The fatty acid may be of natural or synthetic origin, preferably natural origin. Some fatty acids are abundant in vegetable and animal fats. Non-limiting examples include coconut oil, palm kernel oil, sunflower oil, rice bran oil, safflower oil, sesame oil, groundnut oil, palm oil, olive oil, soybean oil, grape seed oil, linseed oil, soybean oil, tallow, tall oil, legume oils, cocoa butter, shea butter and mixtures thereof. Preferably the fatty acid is coconut oil, palm kernel oil or mixtures thereof, more preferably coconut oil. It is also possible to use hydrogenated fatty acids such as hydrogenated coconut oil or hydrogenated palm kernel oil, preferably hydrogenated coconut oil.

Polyethylene glycol (PEG) is a compound having the formula H—(O—CH₂CH₂)ₙ—OH. It is also known as polyethylene oxide or Macrogol. The average number of the ethylene oxide repeating units (n) can vary and can range, e.g., from about 6 to about 200, preferably about 6 to about 100, more preferably about 6 to about 40, even more preferably about 20 to 40. In some embodiments, the PEG has about 32 ethylene glycol units (i.e. PEG-32) on average. The PEG of the monoester and/or diester of fatty acid and PEG may include PEG chains of different lengths, i.e. different amounts of ethylene glycol (ethylene oxide, EO) units. The monoester and/or diester of fatty acid and PEG disclosed herein may have a PEG having a molecular weight of from about 300 to about 2000 g/mol (Da). In some embodiments, the PEG has a molecular weight of about 260 to about 10,000 g/mol, preferably about 260 to about 4,400 g/mol, more preferably about 260 to about 1,800 g/mol, even more preferably about 880 to 1,800 g/mol. In some embodiments, the PEG has a molecular weight of about 1500 g/mol.

In some embodiments, the excipient comprises a monoester and diester of a fatty acid and PEG, each having a PEG with about 32 ethylene glycol units and a fatty acid as defined above. The fatty acid can be caprylic acid (C₈), capric acid (C₁₀), lauric acid (C₁₂), myristic acid (C₁₄), palmitic acid (C₁₆), and/or stearic acid (C₁₈). In some embodiments, a preferred fatty acid comprises lauric acid (C₁₂). In some embodiments the mono-, di- and triglycerides comprise glycerol and a fatty acid component which includes caprylic acid (C₈), capric acid (C₁₀), lauric acid (C₁₂), myristic acid (C₁₄), palmitic acid (C₁₆), and/or stearic acid (C₁₈), preferably including lauric acid (C₁₂).

The excipient is generally non-aqueous. It can be in the form of a semisolid waxy material which is amphiphilic in nature. Due to its structure the excipient is surface active.

The excipient is typically a mixture so that there is a range in which it melts it rather than a specific melting point. The melting range of the excipient can be in the range of about 33° C. to about 64° C., preferably about 35° C. to about 55° C., more preferably about 42.5° C. to about 47.5° C., even more preferably about 44° C. If the excipient contains a mixture of compounds it will typically exhibit a melting range rather than a defined melting point. In some embodiments, the excipient disclosed herein contains a mixture, e.g. mono-, di- and triglycerides and PEG fatty acid esters and exhibits a melting range with an onset melting temperature of about 38° C. and a peak melting temperature of about 43° C.

The excipient can also be characterized by its HLB value (hydrophilic-lipophilic balance). The HLB value can, for instance, range from about 1 to about 16, preferably from about 7 to about 14, more preferably about 11 to about 14, or 11 or 14.

The delivery properties (e.g., immediate release, sustained release) can be modified by a skilled person by selecting the appropriate melting point and HLB. In particular embodiments, the excipient and API exists as an emulsion at body temperature.

A preferred type of excipients are polyoxylglycerides. Polyoxylglycerides are, for instance, available under the trade designation Gelucire® such as Gelucire® 43/01 (mono-, di- and triglyceride esters of fatty acids (C₈ to C₁₈ USP “hard fat”), Gelucire® 44/14 (lauroyl polyoxyl-32 glycerides), Gelucire® 48/16 (Polyethylene glycol monostearate), Gelucire® 50/13 (Stearoyl polyoxyl-32 glycerides) and Gelucire® 59/14 (Mixture of lauroyl polyoxyl-32 glycerides and PEG 6000). In a preferred embodiment, the excipient is Gelucire® 44/14.

Information about lauroyl polyoxylglycerides and other related polymers may be found in, for example, Panigrahi, 2017, Jannin, 2009 and Strickley, 2004.

The excipient may be synthesized by any suitable method. In one embodiment, the excipient can be prepared by partial alcoholysis between a glyceride of the fatty acid and the polyethylene glycol, for example partial alcoholysis between optionally hydrogenated coconut oil and/or optionally hydrogenated palm kernel oil and polyethylene glycol such as PEG-32. A possible reaction is shown the following scheme:

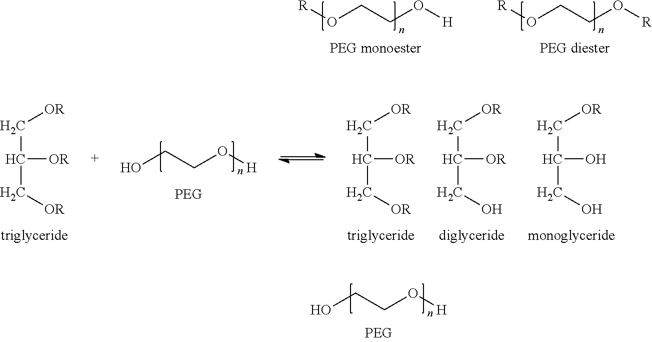

| | |
|---|---|
| ″PEG monoester″ | monoester of a fatty acid and polyethylene glycol |
| ″PEG diester″ | diester of a fatty acid and polyethylene glycol |
| ″triglyceride″ | triglyceride of a fatty acid |
| ″diglyceride″ | diglyceride of a fatty acid |
| ″monoglyceride″ | monoglyceride of a fatty acid |
| ″PEG″ | polyethylene glycol |

In the scheme, R denotes the residue of the at least one fatty acid (i.e., —C(O)—R* with R* being the aliphatic chain of the fatty acid which is optionally substituted by a hydroxy group) and n denotes the number of repeating ethylene oxide units in the PEG. The fatty acid HO—C(O)—R* is as defined above.

In a particular embodiment, the excipient is obtained by polyglycolysis of hydrogenated vegetable oil with PEG, for example optionally hydrogenated coconut oil or optionally hydrogenated palm kernel oil with polyethylene glycol such as PEG-32. In another embodiment, the excipient can be obtained by esterification of polyols with a fatty acid. For instance, esterification of glycerol with a fatty acid, esterification of polyethylene glycol (PEG) with the fatty acid and admixing. Free PEG may be present in the excipient. If desired, further free polyethylene glycol can be added.

The amounts of the compound having the general formula (A) and/or (A*) and the excipient in the pharmaceutical composition are not particularly limited. The pharmaceutical composition can, for instance, comprise about 2 wt % to about 10 wt %, preferably about 3 wt % to about 5 wt %, of the compound having the general formula (A) and/or (A*). The pharmaceutical composition can comprise about 90 wt % to about 98 wt % (preferably about 95 wt % to about 97 wt %) of the excipient, based on 100 wt % of the total pharmaceutical composition.

In some embodiments, the pharmaceutical composition comprises about 90 wt % to about 98 wt %, preferably about 95 wt % to about 97 wt %, of the excipient, which is lauroyl polyoxyl-32 glycerides and about 2 wt % to about 10 wt %, preferably about 3 wt % to about 5 wt %, of the compound having the general formula (A) and/or (A*). In particular embodiments, the pharmaceutical composition comprises about 90 wt % to about 98 wt %, preferably about 95 wt % to about 97 wt %, of the excipient, which is lauroyl polyoxyl-32 glycerides and about 2 wt % to about 10 wt %, preferably about 3 wt % to about 5 wt %, of the compound having the general formula (B) and/or (B*).

The pharmaceutical composition is formulated and dosed in a fashion consistent with good medical practice, taking into account the clinical condition of the individual patient, the site of delivery of the pharmaceutical composition, the method of administration, the scheduling of administration, and other factors known to practitioners. The “effective amount” of the pharmaceutical composition for purposes herein is thus determined by such considerations.

The pharmaceutical compositions disclosed herein are preferably formulated for oral administration and may be in a unit dose in the form of a tablet, capsule, caplet and the like, preferably a capsule.

In one embodiment, the unit dosage form comprises from 5 mg to 100 mg, or from 10 mg to 75 mg or from 10 mg to 50 mg or 10 mg or 30 mg of the compound having the general formula (A), (A*) or a mixture thereof. In particular embodiments, the unit dosage form comprises from 5 mg to 100 mg, or from 10 mg to 75 mg or from 10 mg to 50 mg or 10 mg or 30 mg of the compound having the general formula or (B), (B*) or a mixture thereof.

The pharmaceutical composition of the present invention is for use in medicine. In particular, the pharmaceutical composition of the present invention is for use in the treatment or prevention of a disease associated with protein aggregation and/or a neurodegenerative disease.

The compound having the general formula (A) and/or (A*) can be used for the preparation of a pharmaceutical composition of the present invention, wherein the pharmaceutical composition is for treating or preventing a disease linked to protein aggregation and/or a neurodegenerative disease. In particular embodiments, the compound having general formula or (B) and/or (B*) is used for the preparation of a pharmaceutical composition of the present invention.

In a further embodiment, the present invention is directed to a method of treating or preventing a disease linked to protein aggregation and/or a neurodegenerative disease comprising administering a therapeutically effective amount of a pharmaceutical composition of the present invention to a patient in need thereof. In particular embodiments, the method comprises administering a pharmaceutical composition comprising a compound having general formula or (B) and/or (B*).

The disease linked to protein aggregation is characterized by the presence of an aggregated form of at least one protein or a fragment or derivative thereof, wherein the protein is selected from the group consisting of α-synuclein, prion protein, Abeta (Amyloid beta, Aβ), tau, amyloid precursor protein (APP), superoxide dismutase, immunoglobulin, amyloid-A, transthyretin, beta 2-microglobulin, cystatin C, apolipoprotein A1, TDP-43, islet amyloid polypeptide, ANF, gelsolin, insulin, lysozyme, fibrinogen, huntingtin and ataxin and other proteins with a poly-Q stretch. Preferably α-synuclein, prion protein, Abeta (Amyloid beta, Aβ), and tau, more preferably α-synuclein.

Examples of diseases include, but are not limited to, Parkinson's disease, multiple system atrophy, dementia with Lewy bodies (DLB), prion disease, Alzheimer's disease, frontotemporal dementia, amyotrophic lateral sclerosis, Huntington disease's, spinocerebellar ataxias and other Poly-Q diseases, hereditary cerebral amyloid angiopathy, familial amyloid polyneuropathy, primary systemic amyloidosis (AL amyloidosis), reactive systemic amyloidosis (AA amyloidosis), type II diabetes, injection-localized amyloidosis, beta-2 microglobulin amyloidosis, hereditary non-neuropathic amyloidosis, and Finnish hereditary systemic amyloidosis. Preferably, the disease is Parkinson's disease, multiple system atrophy (MSA), or dementia with Lewy bodies (DLB).

As used herein, the terms “treating” and “method of treatment” as well as different forms thereof include preventative (e.g., prophylactic), curative, or palliative treatment. As used herein, the term “treating” includes alleviating or reducing at least one adverse or negative effect or symptom of a condition, disease or disorder. This condition, disease or disorder may be a neurodegenerative disease, including an α-synucleinopathy, for example MSA, PD, DLB and the like.

The term “administering” means providing to a patient the pharmaceutical composition or unit dose of the present invention.

The term “therapeutically effective amount” refers to the amount of a compound, e.g. a compound having general formula (A) and/or (A*) or general formula (B) and/or (B*) that, when administered, is sufficient to elicit the biological or medical response of a cell, tissue, system, animal, or human that is being sought by a clinician.

The terms “subject” and “patient” are used interchangeably herein, for example, to a mammalian subject, preferably a human or human patient.

The singular forms “a,” “an,” and “the” may refer to plural articles unless specifically stated otherwise.

The term “wt %” refers to a weight percent of the total weight, for example total weight of the pharmaceutical composition. In some embodiments, a unit dosage, comprises about 2 wt % to about 10 wt %, of the compound disclosed herein, based on 100 wt % of the total pharmaceutical composition of the unit dosage. In specific embodiments, the pharmaceutical composition comprises about 3 wt % to about 5 wt % of a compound disclosed herein, and about 95 wt % to about 97 wt % of the excipient, based on 100 wt % of the total pharmaceutical composition.

As used herein, reference to a total weight of a dosage form in the case of a capsule, refers to the total weight of the capsule contents, excluding the weight of the capsule itself. The type of capsule is not limiting and may be manufactured from natural or synthetic materials, including gelatin or hydroxypropyl methylcellulose (HPMC).

As used herein, the term “once daily” and “QD” refer to once a day dose administration, about once every 24 hours. As used herein, the term “twice daily” and “BID” refer to twice a day dose administration, typically once in the morning and once in the evening.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “combination therapy” refers to the administration of two or more therapeutic agents to treat a therapeutic disorder described herein. Such administration encompasses co-administration of these therapeutic agents in a substantially simultaneous manner, such as in a single dosage form having a fixed ratio of active ingredients or in multiple, separate dosage forms for each active ingredient. In addition, such administration also encompasses use of each type of therapeutic agent in a sequential manner. In either case, the treatment regimen will provide beneficial effects of the drug combination in treating the disorders described herein. A non-limiting example is in the treatment of Parkinson's disease, wherein the compound having the general formula (A) and/or (A*) or general formula (B) and/or (B*) may be administered in combination with, for example, a dopaminergic active pharmaceutical ingredient (API) or antiparkinsonian API, which increases dopamine-related activity in the brain. Such API may be chosen, for example, from dopamine precursors, dopamine agonists, inhibitors of the degradation of dopamine and/or of dopamine agonists, dopa decarboxylase inhibitors, and other APIs (Cacabelos, 2017). Therapeutic compounds that may be administered with the compound having the general formula (A) or (A*) include levo-DOPA, carbidopa, opicapone, rasagiline and the like.

The pharmacokinetic (PK) terms “Tmax”, “max”, “T½”, “AUC₀₋ₜ”, “AUC₀₋₂₄”, and “AUC_{0-inf}” are terms known in the art. Tmax refers to Time of maximum observed concentration, Cmax refers to maximum observed concentration, T½ refers to the time taken for Cmax to drop to half, AUC₀₋ₜ is the area under the plasma concentration versus time curve from time zero to a set time, t; AUC₀₋₂₄ is the area under the plasma concentration versus time curve from time zero to 24 hours, and AUC_{0-inf} is the area under the concentration-time curve from time zero to infinity. In particular embodiments, the compound of general formula (B) and/or (B*) (i.e. anle138b) is measured in the blood plasma and PK parameters disclosed herein refer to measurements of anle138b.

The term “bioavailability” or abbreviated as “BA”, refers to the amount of a drug, i.e. the compound having the general formula (A) or (A*), absorbed in the body and has a pharmaceutical effect. For example, bioavailability may refer to the fraction of drug in systemic circulation following administration to a subject or patient under fed or fasted state.

It is well understood in the art of pharmaceutical formulation that the pharmacokinetic (PK) performance of some compositions is affected by the presence or absence of food in the gastrointestinal (GI) system. A “food effect study” is typically undertaken to observe the effect of food on drug bioavailability (BA) between fed and fasted treatments. Accordingly, administration of an oral dosage form exhibiting a food effect may preferably be made under “fasted” conditions, for example 1 hour before or 2 hours after a meal. As used herein, the term “without regard to food” or “without regard to meals” means that the human exposure to the drug is not substantially affected by food and that the drug product, i.e. pharmaceutical composition of the present invention, may be administered irrespective of the human subject's fed state. In some embodiments, wherein a single dose of a compound of formula (B), (B*) or a mixture thereof is administered to healthy subject, the ratio of fasted AUC(0-24) to fed AUC(0-24) of anle138b is less than 1.5, preferably less than 1.4.

The US FDA requirements for a food effect study, and definition of a fasted state and “fed state” may be found at https://www.regulations.gov/document?D=FDA-2001-D-0040-0003 (Jul. 22, 2020), the entirety of which is incorporated herein by reference.

For the foregoing embodiments, each embodiment disclosed herein is contemplated as being applicable to each of the other disclosed embodiments. For example, the features recited in the composition embodiments can be used in the use embodiments described herein and vice versa.

Having described the disclosure with reference to certain preferred embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### EXAMPLES

### Example 1.1: anle138b Formulation Studies

Anle138b is a lipophilic compound. Studies were performed to assess approaches to increase solubility of anle138b for formulation development.

The formulations were prepared as gelatin capsules and were tested for drug loading, in vitro dissolution in bio-relevant dissolution media 0.1N HCl, simulated gastric fluid (SGF) and Fasted State Simulated Intestinal Fluid (FaSSIF) and in vivo (PK in rats).

In an initial excipient screen, three formulations in which anle138b exhibited solubility were prepared. The composition of the formulations and dissolution data from a 0.1N HCl solution are provided in Table 1.

TABLE 1
anle138b test formulations

| Vehicle | Formulation | Loading | 0 h* | 2 h* | 4 h* |
|---|---|---|---|---|---|
| # | (% each ingredient) | (mg) | % | % | % |
| 1 | PEG400 + Kolliphor ® RH40 + | 25 | 94.7 | 92.5 | 84.1 |
| | TPGS + ethanol (42:42:7:9) | | | | |
| 1 | PEG400 + Kolliphor ® RH40 + | 50 | 91.0 | 43.8 | 12.3 |
| | TPGS + ethanol (42:42:7:9) | | | | |
| 2 | Transcutol + Solutol HS + | 25 | 93.2 | 90.2 | 87.9 |
| | TPGS (20:60:20) | | | | |
| 2 | Transcutol + Solutol HS + | 50 | 91.0 | 76.6 | 41.4 |
| | TPGS (20:60:20) | | | | |
| 3 | PEG400 + Caprylol ® 90 + | 50 | 94.5 | 94.2 | 47.5 |
| | Kolliphor ® RH40 (35:20:45) | | | | |

*Percent release of anle138b from formulation in 0.1N HCl dissolution solution, h = hour(s)
Caprylol ® 90 = propylene glycol monocaprylate (type II);
Kolliphor ® RH40 = PEG-40 Hydrogenated Castor Oil;
Solutol = Polyethylene glycol (15)-hydroxystearate;
TPGS = vitamin E, D-α-tocopheryl polyethylene glycol succinate;
Transcutol = Diethylene glycol monoethyl ether.

Vehicles 1, 2, and 3 were selected as having good solubility and were further tested for dissolution in 0.1N HCl medium. Dissolution data show that Vehicle 1 exhibited slow dissolution at a 25 mg dose and too fast a dissolution at the 50 mg dose. Vehicle 2 exhibited slow dissolution at the 25 mg dose and too fast a dissolution at the 50 mg dose. Vehicle 3 exhibited a good dissolution profile for the 50 mg dose and was further tested in simulated gastric fluid (SGF) and Fasted State Simulated Intestinal Fluid (FaSSIF). In both media, the sample started to exhibit large flakes which increased over time and may account for the observed rapid release profile after 60 minutes.

Tables 2A and 2B, provide the dissolution rates of formulation with vehicle 3 as the mean of three samples in SGF and FaSSIF respectively.

TABLE 2A
Dissolution of anle138b vehicle 3 formulation in SGF
50 mg/mL anle138b in Vehicle 3. % release in SGF

| Time (min) | Vessel 1 | Vessel 2 | Vessel 3 | Mean | RSD % |
|---|---|---|---|---|---|
| 5 | 29.293 | 43.893 | 66.454 | 46.547 | 40.222 |
| 15 | 59.634 | 59.182 | 70.950 | 63.255 | 10.541 |
| 30 | 84.425 | 79.404 | 84.882 | 82.904 | 3.666 |
| 45 | 72.357 | 93.465 | 41.047 | 68.956 | 38.247 |
| 60 | 72.765 | 86.934 | 58.712 | 72.804 | 19.382 |
| 90 | 47.701 | 50.821 | 49.848 | 49.457 | 3.228 |
| 120 | 51.712 | 49.143 | 39.873 | 46.909 | 13.276 |
| infinity | 45.141 | 49.329 | 35.425 | 40.238 | 12.298 |

TABLE 2B
Dissolution of anle138b vehicle 3 formulation in FaSSIF
50 mg/mL anle138b in Vehicle 3. % release in FaSSIF

| Time (min) | Vessel 1 | Vessel 2 | Vessel 3 | Mean | RSD % |
|---|---|---|---|---|---|
| 5 | 94.030 | 114.596 | 108.047 | 105.557 | 9.954 |
| 15 | 106.864 | 111.794 | 101.109 | 106.589 | 5.017 |
| 30 | 107.077 | 112.012 | 95.475 | 104.855 | 8.096 |
| 45 | 73.405 | 91.249 | 56.471 | 73.708 | 23.594 |
| 60 | 51.508 | 87.395 | 47.848 | 62.250 | 35.105 |
| 90 | 66.738 | 76.173 | 41.934 | 61.615 | 28.703 |
| 120 | 40.946 | 44.224 | 31.375 | 38.848 | 17.185 |
| infinity | 37.827 | 40.422 | 31.784 | 36.677 | 12.084 |

Visual observations in both media (SGF and FaSSIF): the sample started to changed appearance from a fine suspension to large flake/lumps which increased overtime after 45-60 minutes of the dissolution run. This is in line with the drop in % claim seen after this time point.

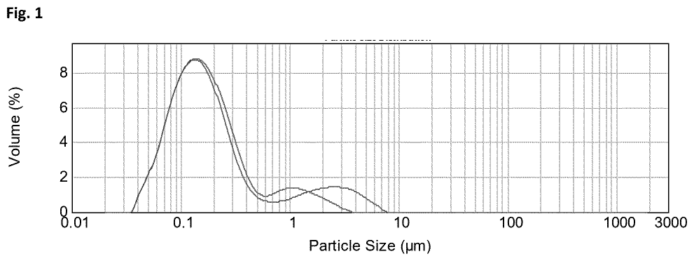

In a second approach, increasing solubility by nanomilling the API was tested. The vehicle was prepared by dissolving 1% w/v of HPMC Pharmacoat 603 (Shin-Etsu) and 0.25% w/v Sodium Lauryl Sulphate (SLS) in pure water; anle138b was added and the suspension underwent 3 consecutive cycles (99 minutes each) of milling performed with Retsch Mill MM200 using 0.6 mm diameter Yttrium Zirconium beads, after which time the resultant suspension was recovered and maintained under constant magnetic stirring until dose administrations. Average particle size distribution (PSD) of nanomilled sample is as follows: D(10) 0.07 um, D(50) 0.156 um, and D(90) 1.57 um. FIG. 1 shows the particle size distribution graph of two batches of nanomilled formulation.

Tables 3A and 3B, provide the dissolution rates of the nanomilled formulation as the mean of three samples in SGF and FaSSIF respectively.

TABLE 3A
Dissolution of anle138b nanomilled formulation in SGF
50 mg/mL anle138b nanomilled formulation release in SGF

| Time (min) | Vessel 1 | Vessel 2 | Vessel 3 | Mean | RSD % |
|---|---|---|---|---|---|
| 5 | 1.195 | 1.950 | 1.928 | 1.931 | 0.916 |
| 15 | 1.1663 | 1.581 | 1.650 | 1.631 | 2.702 |
| 30 | 1.226 | 1.288 | 1.284 | 1.631 | 2.702 |
| 45 | 1.105 | 0.925 | 0.955 | 0.955 | 9.692 |
| 60 | 0.907 | 0.319 | 0.928 | 0.718 | 48.148 |
| 90 | 0.705 | 0.514 | 0.610 | 0.610 | 15.664 |
| 120 | 0.637 | 0.599 | 0.516 | 0.584 | 10.596 |
| infinity | 0.884 | 0.469 | 0.431 | 0.595 | 42.257 |

TABLE 3B
Dissolution of anle138b nanomilled formulation in FaSSIF
50 mg/mL anle138b in Vehicle 3. % release in FaSSIF

| Time (min) | Vessel 1 | Vessel 2 | Vessel 3 | Mean | RSD % |
|---|---|---|---|---|---|
| 5 | 8.656 | 8.607 | 8.843 | 8.702 | 1.433 |
| 15 | 8.826 | 8.848 | 8.665 | 8.779 | 1.138 |
| 30 | 8.972 | 9.240 | 9.031 | 9.081 | 1.550 |
| 45 | 9.027 | 8.980 | 8.911 | 8.973 | 0.653 |
| 60 | 9.040 | 8.818 | 9.000 | 8.953 | 1.321 |
| 90 | 9.593 | 9.371 | 9.388 | 9.451 | 1.308 |
| 120 | 9.201 | 9.334 | 9.370 | 9.302 | 0.957 |
| infinity | 9.287 | 9.242 | 9.399 | 9.310 | 0.868 |

The nanomilled formulation exhibits a slow and steady release profile that releases less than 10% API after 2 h. This was considered to be an unacceptable release profile. Drug product appears as a fine suspension throughout the dissolution run.

In a third approach, anle138b was formulated in a single solubilizer, lauroyl polyoxyl-32 glycerides, also known by its tradename Gelucire® 44/14, a mixture of one monoester and/or diester of a fatty acid, mono-, di- and tri-glycerides of a fatty acid and free PEG.

Tables 4A and 4B provide the dissolution rates of the anle138b lauroyl polyoxyl-32 glycerides formulation in capsules as the mean of three samples in SGF and FaSSIF respectively.

TABLE 4A
Dissolution of anle138b lauroyl polyoxyl-32 glycerides in SGF
50 mg/mL anle138b lauroyl polyoxyl-32 glycerides in SGF

| Time (min) | Vessel 1 | Vessel 2 | Vessel 3 | Mean | RSD % |
|---|---|---|---|---|---|
| 5 | 6.335 | 6.987 | 11.501 | 8.274 | 34.001 |
| 15 | 35.601 | 54.012 | 46.824 | 45.479 | 20.401 |
| 30 | 71.137 | 93.224 | 72.555 | 78.972 | 15.655 |
| 45 | 91.814 | 109.075 | 71.892 | 90.927 | 20.464 |
| 60 | 98.409 | 111.684 | 81.575 | 97.223 | 15.521 |
| 90 | 99.598 | 108.475 | 85.179 | 97.751 | 12.028 |
| 120 | 93.646 | 99.305 | 80.709 | 92.422 | 12.065 |
| infinity | 88.403 | 99.305 | 79.128 | 88.945 | 11.355 |

TABLE 4B
Dissolution of anle138b lauroyl polyoxyl-32 glycerides in FaSSIF
50 mg/mL anle138b lauroyl polyoxyl-32 glycerides in FaSSIF

| Time (min) | Vessel 1 | Vessel 2 | Vessel 3 | Mean | RSD % |
|---|---|---|---|---|---|
| 5 | 8.853 | 8.059 | 9.341 | 8.751 | 7.392 |
| 15 | 58.037 | 63.880 | 60.453 | 60.790 | 4.830 |
| 30 | 96.011 | 104.515 | 102.318 | 100.948 | 4.373 |
| 45 | 107.572 | 113.679 | 113.458 | 111.570 | 3.105 |
| 60 | 111.606 | 113.454 | 113.458 | 112.872 | 0.973 |
| 90 | 112.110 | 113.409 | 113.194 | 112.904 | 0.617 |
| 120 | 111.288 | 112.338 | 111.845 | 111.823 | 0.470 |
| infinity | 112.159 | 110.403 | 111.572 | 111.378 | 0.803 |

Tables 4A and 4B show that the capsules dissolve after around 15 to 30 minutes of the dissolution runs and provide a steady release profile in biologically relevant simulated fluids (simulated gastric fluid (SGF) and fasted state intestinal fluid (FaSSIF)) and was considered to be suitable for clinical use.

The formulation comprising lauroyl polyoxyl-32 glycerides (“Gelucire® 44/14”) was selected for further pre-clinical and clinical development. The studies resulted in the identification of a formulation consisting of a semisolid in capsule formulation in size 00 capsule shells (2 dose strengths, 10 mg and 30 mg anle138b per capsule, respectively, and 1 placebo) with lauroyl polyoxyl-32 glycerides (Gelucire® 44/14) as the excipient.

### Example 1.2 Formulation Study in Rat

A rat study was undertaken to assess the pharmacokinetics of anle138b in male Sprague Dawley rats (n=3/arm) following single oral administration of different formulations of anle138b, as follows, in Table 5.

TABLE 5
anle138b formulation used in rat study

| | Anle138 | |
|---|---|---|
| Formulation | (10 mg/kg; 2 mL/kg) | Composition |
| A | | PEG400 (35%) (Sigma) |
| | | Capryol ® 90 (20%) (Gattefosse) |
| | | Kolliphor ® RH40 (45%) (Sigma) |
| B | Nanomilled | Suspension in HPMC Pharmacoat |
| | | 603 (1% w/v) (Shin-Etsu) |
| C | | PEG400 (Sigma) |
| D | | Lauroyl polyoxyl-32 glycerides |
| | | (Gattefosse) |

Capryol ® 90 = propylene glycol monocaprylate (type II);
Kolliphor ® RH40 = PEG-40 Hydrogenated Castor Oil.

Preparation of Formulation A: The vehicle was prepared by weighing Kolliphor® RH40 (Sigma Aldrich; 45% of final volume); PEG400 (Sigma Aldrich; 35% of final volume) and Caprylol 90 (Gattefosse; 20% of final volume). The mixture was stirred and warmed to approximately 50° C. (using a thermostatically controlled bath) for ca. 15 minutes until a clear liquid was obtained. The anle138b was added to vehicle maintained under continuous agitation at 50° C. The mixture was stirred for a further 15 minutes and sonicated for 10 minutes until a visually clear solution was obtained.

Preparation of Dose Formulation B: The vehicle was prepared by dissolving 1% w/v of HPMC Pharmacoat 603 (Shin-Etsu) and 0.25% w/v Sodium Lauryl Sulphate (SLS) in pure water; anle138b was added and the suspension underwent 3 consecutive cycles (each of 99 minutes) of milling performed with Retsch Mill MM200 using 0.6 mm diameter Yttrium Zirconium beads, after which time the resultant suspension was recovered and maintained under constant magnetic stirring until dose administrations. Average particle size distribution (PSD) of nanomilled sample is as follows: D(10) 0.07 μm, D(50) 0.156 μm, and D(90) 1.57 μm.

Preparation of Dose Formulation C: The anle138b was added to PEG400 (Sigma Aldrich) under constant magnetic stirring until complete dissolution of the test material was obtained.

Preparation of Dose Formulation D: The vehicle (Gattefosse; lauroyl polyoxyl-32 glycerides) was warmed to at least 20° C. above its melting point and anle138b was added under constant magnetic stirring. The formulation was maintained at 40° C. in a thermostatically controlled water bath and syringes and cannulae in the treatment room were also pre-assembled and warmed prior to dosing.

Anle138b was formulated on the day of dosing, except for formula C, which was prepared one day in advance.

Male Sprague Dawley (SD) rats (n=3/formulation) sourced from Charles River Italia were kept for 5 days acclimatization. Feeding regimen was ad libitum except on the day of dosing when the food was supplied 4 hours post dosing, having been removed the evening beforehand. Animals were orally dosed with anle138b (10 mg/kg) formulated as above. An individual serial plasma profile was drawn from each animal over a period of 24 hours after dosing.

Actual body weights were recorded on the day of dose administration. Dose volumes were adjusted taking into account the weight of the animal at the time of dose administration.

The dose formulations were administered orally via gastric gavage (2 ml/kg); any remaining formulation after dosing was discarded.

After oral administration, blood samples were collected from the tail vein of each rat at the time-points detailed: Pre dose, 0.5, 1, 2, 4, 6, 8 and 24 hours after dosing

Approximately 150 μL blood was collected into tubes containing anticoagulant (K3 EDTA), placed on crushed wet-ice and then centrifuged (2000 g at +4° C. for 10 minutes) as soon as practicable and in any case within 1 hour. The resultant plasma was separated from the erythrocyte pellet and then transferred to uniquely labeled clear polypropylene tubes and frozen immediately over solid carbon dioxide or in a freezer at nominally −20° C.

PK profiling was performed by non-compartmental analysis using Phoenix™ WinNonlin. All computations utilized the nominal sampling times. Where feasible, the systemic exposure to anle138b was determined by calculating the area under the plasma concentration time curve (AUC) from the start of dosing to the last quantifiable time-point (AUC0-t, t=8 h) using the linear-logarithmic trapezoidal rule. The maximum observed peak plasma concentration (Cmax) and the time at which it was observed (Tmax) were determined by inspection of the observed data.

The relative bioavailability (Free %) of anle138b between dose formulations was calculated by comparing systemic exposure (AUC0-t), and are reported rounded to at least 2 significant figures. All dose levels, plasma concentrations and pharmacokinetic parameters are given in terms of parent compound anle138b.

Results: Following a single oral administration of anle138b 10 mg/kg to male rats, anle138b was quantifiable in the plasma of all animals up to 8 hours after dosing. Tmax occurred between 2 and 8 hours post dosing. Notable differences in systemic exposure to anle138b, as mean Cmax and AUC0-t, were observed between the four formulations evaluated. PK values (mean and ranges) are provided in Table 6.

TABLE 6
PK date from rat formulation study

| | AUC0-t (ng*h/mL) | Cmax (ng/mL) | Tmax (h) |
|---|---|---|---|
| Formula | Mean / Range | Mean / Range | Mean / Range |
| A | 3440 | 673 | 4.00 |
| | 2820-3810 | 567-751 | 2.00-4.00 |
| B | 861 | 244 | 2.00 |
| | 791-947 | 224-279 | 2.00-2.00 |
| C | 1700 | 417 | 6.00 |
| | 476-2770 | 221-647 | 4.00-8.00 |
| D | 4800 | 1050 | 4.00 |
| | 3850-5390 | 902-1160 | 4.00-4.00 |

AUC0-t = area under the plasma concentration-time curve (AUC) from the start of dosing (0) to the last quantifiable time point (t) which was 8 hours.

Differences in systemic exposure to anle138b, as mean Cmax and AUC0-8h, were observed between the four formulations. Formulations A and D performed the best in terms of AUC and Cmax, with administration of formulation D, resulting in an anle138 mean AUC0-8h approximately 5.6, 3 and 1.4-fold higher than the values obtained after dosing of formulations B, C and A respectively.

Using formulation A as a reference point, the relative bioavailability (Frel %) of anle138b formulated as B vs A (ref) was approximately 25%, C vs A (ref) was approximately 49% and D vs A (ref) was approximately 140%. The PK rat data shows that following a single administration, the formulae A and D exhibited better AUC and higher Cmax than a suspension of nanomilled anle138b or anle138b in PEG400. Formula D (Gelucire 44/14) exhibited the more favorable AUC and Tmax overall and was selected for further development and testing.

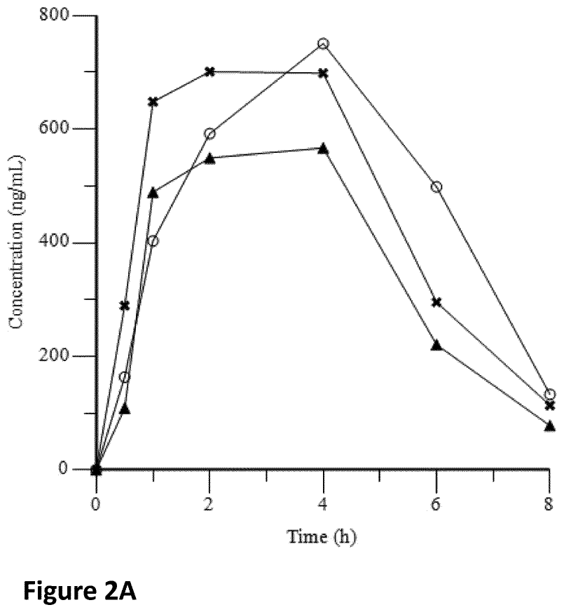

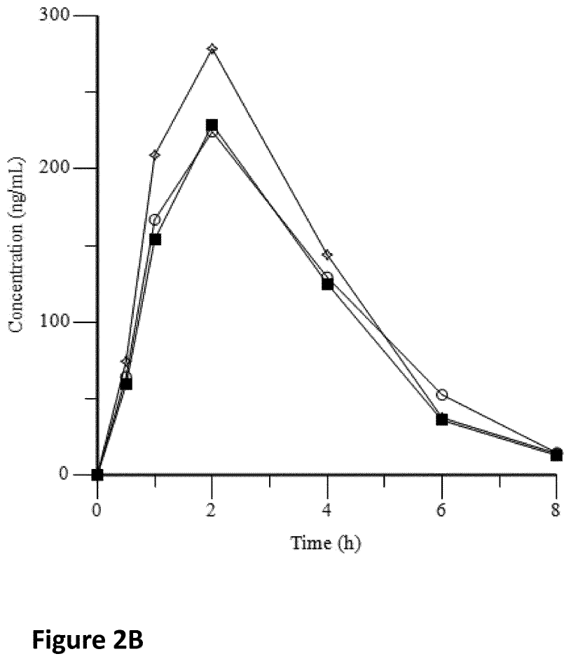

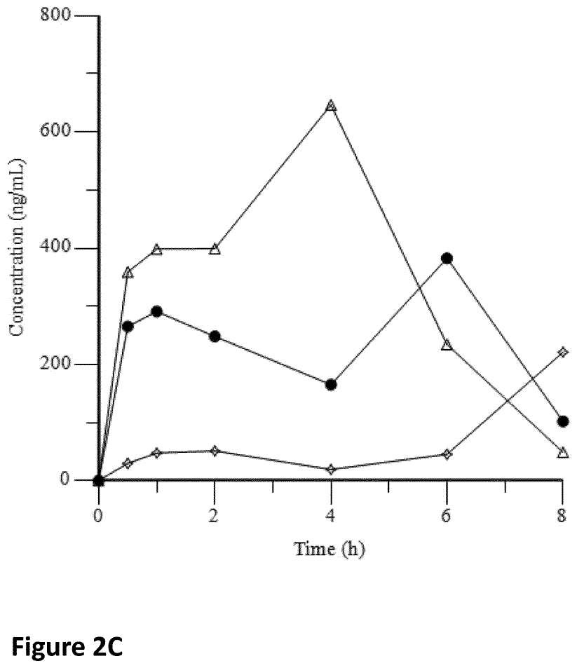

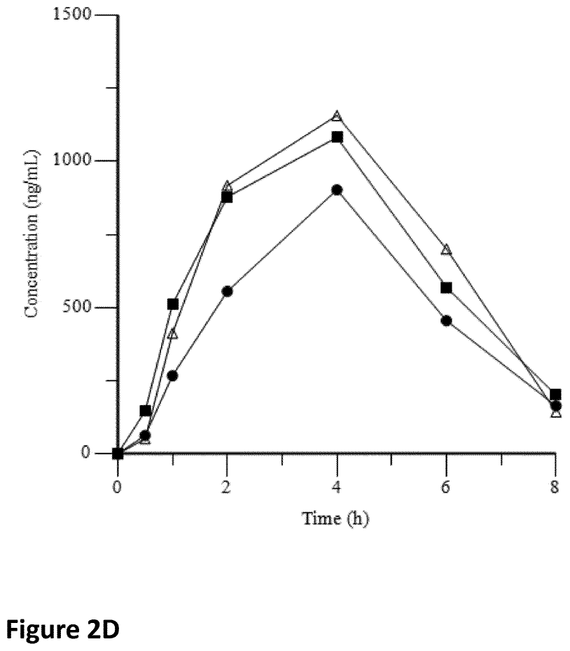

FIGS. 2A-2D are graphs showing PK Cmax profiles of each of the 3 animals in each group. FIG. 2A represents PK results with formulation A, FIG. 2B represents PK results with formulation B, FIG. 2C represents PK results with formulation C, FIG. 2D represents PK results with formulation D.

### Example 1.3 Phase 1 Drug Product (DP)

Table 7 provides the anle138b phase 1 DP ingredients

TABLE 7
10 mg and 30 mg drug product ingredients.

| Ingredient | | 10 mg | 30 mg |
|---|---|---|---|
| Anle138b | API | 10.0 mg | 30.0 mg |
| Lauroyl polyoxyl-32 glycerides | Ph. Eur. | 250.0 mg | 750.0 mg |
| Total | | 260.0 mg | 780.0 mg |
| Hard gelatin capsules, size 00 | Ph. Eur. | 1 ea | 1 ea |
| Gelatin | Ph. Eur. | q.s. | q.s. |
| Purified Water* | Ph. Eur. | N/A | N/A |

*water evaporates during capsule band drying

The DP was filled into white high-density polyethylene (HDPE) bottles closed with tamper evident caps.

Evaluation of Solubility in Lauroyl Polyoxyl-32 Glycerides: The visual solubility of API in molten lauroyl polyoxyl-32 glycerides was assessed at 55° C. Six solutions at different concentrations (up to saturation) were prepared and then cooled down to room temperature. The concentration of 50 mg (API)/g of lauroyl polyoxyl-32-glycerides was considered as a reference start.

Preparation of Capsules and Stressed Stability Study: Two API doses were selected (10 mg and 30 mg) and corresponding solutions of API in molten lauroyl polyoxyl-32 glycerides were prepared to be filled into capsules. These capsule prototypes were banded and then stored at 40° C./75% RH and 50° C./ambient RH condition and tested for assay and impurities after 15 days and 30 days to check for any incompatibility (RH: relative humidity).

Scale-up with Technical Batch Manufacture: The manufacture of semisolid in capsule formulations (two dose strengths) was scaled up to a batch size representative of clinical manufacture (1000-3000 units). A placebo batch was also prepared.

Preparation of solution: The vehicle was prepared by melting the lauroyl polyoxyl-32 glycerides at 55° C. API was then added and mixed until complete solubilization.

Preparation of capsules: The solution (pure molten vehicle for placebo) was filled into capsules by a HIBAR-P0450 machine at an appropriate fill weight and then capsules were banded using the BONAPACE-BD3000 banding machine using an appropriate solution of gelatin in water. After banding, visually damaged and/or unsuitably sealed capsules were discarded.

Capsules were left to dry for at least 24 h and then checked for integrity of shell and banding and for possible leaking under vacuum (T=55° C. and P<100 mbar; Heraeus VT6130M). Capsules showing material leaks were discarded.

Formal Stability Study on Technical Batch: Stability testing of two active batches and one placebo batch, packaged in typical primary packaging (i.e. HDPE bottle packs with desiccant) was conducted as detailed within the schedule shown below, in Table 8.

TABLE 8
Stability test of DP

| Storage | Time (Months) | | | | | | | | |
|---|---|---|---|---|---|---|---|---|---|
| conditions | 0 | 1 | 3 | 6 | 9 | 12 | 18 | 24 | 36 |
| 5° C. | T* | — | — | (T) | — | (T) | — | (T) | |
| 25° C./60% RH | | T | T | T | (T) | (T) | (T) | (T) | T |
| 30° C./65% RH | | (T) | (T) | (T) | (T) | (T) | — | (T) | |
| 40° C./75% RH | | T | T | T | — | — | — | — | |

Key:
T = Tested for appearance, and drug-related impurities (by single HPLC method), dissolution, and water content by KF titration.
(T) = Optional testing (i.e. storage only).
T* = Assumes QC release testing results are used for initials (i.e. stability study is set down within 30 days of QC release testing).

The anle138b capsules (DP test and placebo) were studied in a 36 month long ICH compliant stability study under long-term conditions (at 25° C./60% RH) and 6 months under accelerated conditions (at 40° C./75% RH). Drug product stability is tested using standard assays including content uniformity, impurities, appearance, water content, dissolution.

The anle138b DP as described herein is stable for at least 18 months at 25° C./60% RH, preferably for at least 24 months and more preferably for at least 36 months.

For subsequent clinical trials, a drug product using hydroxypropyl methylcellulose (HPMC) capsules was manufactured as described above substituting the HPMC capsules for the gelatin capsules.

### Example 2: Human Studies

Example 2.1 Phase 1: Safety, tolerability, and pharmacokinetics of anle138b: a first-in-human (FIH) randomized, double-blind, placebo-controlled phase 1 trial.

Anle138b was studied in a single-centre, double-blind, randomised, placebo-controlled single ascending dose (SAD) and multiple ascending dose (MAD) study in healthy subjects. Eligible participants were randomly assigned (1:1 for sentinel subjects and 1:5 for main group) to placebo or anle138b (dose ranging from 50 mg to 300 mg per day), respectively. In addition, the effect of food on the pharmacokinetics (PK) of anle138b in healthy subjects was examined at doses of 150 mg per day (FES, food effect study). Participants were randomized to treatment sequence (fed→fasted) or (fasted→fed). Treatment for the SAD, MAD and FES arms of the study was administered orally in hard gelatin capsules containing either 10 mg or 30 mg of anle138b with excipient (i.e., lauroyl polyoxyl-32 glycerides) or excipient only. The primary endpoints were safety and tolerability, the secondary endpoint was pharmacokinetics. Data from all randomized individuals were evaluated. [Clinicaltrials.gov-identifier: NCT04208152. EudraCT-number: 2019-004218-33]

Findings: 196 healthy volunteers were screened and 68 participants were enrolled. Of these, all completed the study per protocol. Adverse events in this healthy volunteer trial were mostly mild and all fully recovered or resolved. No abnormal trend was seen in any system organ class. The study drug was safe and well tolerated at all dose levels and reached significantly higher plasma levels in humans than those required for full therapeutic efficacy in MI2 mice, a recently established-synucleinopathy rodent model (Wegrzynowicz, 2019; Levin 2022).

### Methods

Study design: A single-centre, double-blind, randomised, placebo-controlled single ascending dose (SAD) and multiple ascending dose (MAD) study of anle138b in doses of up to 300 mg per day in healthy subjects. The effect of food (FES) on the PK of anle138b in healthy subjects was examined using doses of 150 mg. Participants were recruited from Quotient Sciences (the “CRO”, Nottingham, UK) volunteer database. Approvals from the ethical review board and from the Medicines and Healthcare products Regulatory Agency (UK) were obtained. This study was conducted in accordance with the protocol and with the following legislation: The updated version of the international Council for Harmonisation Good Clinical Practice (GCP) including the integrated Addendum E6, the Medicines for Human Use (Clinical Trials) Regulations including amendments No. 1928, 2984 and 941. In addition, the study was performed according to the ethical principles outlined in the World Medical Association Declaration of Helsinki and its amendments. The study data was monitored. An independent study monitor was appointed to verify that the study was conducted in accordance with current GCP, regulatory requirements, the protocol and that the data were authentic, accurate and complete.

Participants were healthy volunteers aged 18 to 55 and able to understand the nature of the study and any risks involved in participation. They needed to be willing to cooperate and comply with the protocol restrictions and requirements and be capable and willing to give written informed consent. The study included healthy male volunteers and healthy female volunteers with no childbearing potential. Eligible participants needed to have a body mass index of (BMI) of 18.5 to 30.0 kg/m² at screening. For participation in the food effect study, subjects had to be able to eat 90% of the US Food and Drug Administration (FDA)-approved high-fat breakfast, including bacon.

Randomisation and masking: Eight participants were enrolled per dosing cohort in the SAD and MAD parts of the study. Participants were randomly assigned to placebo (N=2) or to anle138b (N=6). Sentinel subjects were randomized 1:1 and the main group 1:5 for placebo or anle138b, respectively. A computer-generated randomisation schedule was used. All participants and study personnel directly interacting with participants were blinded to treatment assignment. The medication kits were numbered in a consecutive manner.

In FES, participants (N=12) were unblinded to treatment as this cohort did not include placebo, but were randomized in a 1:1 ratio to the sequences of treatment in a 2-way crossover design. Hence, 6 volunteers were randomized to the treatment sequence with prandial state first fed and then fasted, the remaining 6 volunteers to the fasted state first and then fed.

Safe starting dose & exposure limit justification: This trial followed the recommendations of the EMA (European Medicines Agency) (EMEA/CHMP/SWP/28367/07 Rev. 1; 20 Jul. 2017) and the FDA (Food and Drug Administration) (Guidance for Industry: Estimating the Maximum Safe Starting Dose in Initial Clinical Trials for Therapeutics in Adult Healthy Volunteers. U.S. Department of Health and Human Services Food and Drug Administration Center for Drug Evaluation and Research (CDER) July 2005) for dose finding in a first-in-human study. The safe starting dose was set based on toxicity studies in animals. In a 28-day toxicity study in rats, the no-observed-adverse-effect-level (NOAEL) was determined to be 50 mg/kg/day (human equivalent dose of 8.1 mg/kg). The dose range of the current trial was designed to start at 50 mg. Escalation between doses was planned to be flexible depending on emerging results but not to exceed an increment of 2-fold.

Procedures: The trial medication was produced by Aptuit, Italy. Anle138b in a capsule was prepared as described in Example 1.3, supra.

Subjects were screened for enrolment in the study up to 28 days before dosing, were admitted in the morning on the day before dosing (day −1) to the clinical site, and remained on site until 48 h post last dose. A post study follow-up visit took place 5 to 7 days post last dose for safety & well-being monitoring. Screening of the volunteers included full physical examination, taking medical history and reviewing medical report, checking body weight and height to calculate body mass index (BMI), safety procedures such as safety bloods (haematology, clinical chemistry & virology, serum pregnancy test), 12 lead ECGs, vital signs (blood pressure, heart rate, and oral temperature), carbon monoxide breath tests, drug screen for drug of abuse, alcohol breath tests and urinalysis.

In SAD, subjects received a single dose of anle138b or placebo after fasting from all food and drink (except water) for a minimum of 8 h prior to dosing. The dosing in the four SAD-cohorts were 50 mg, 100 mg, 200 mg and 300 mg of free anle138b equivalent, QD, cohorts A, B, C and D respectively. In the MAD part, participants received anle138b or placebo once daily (QD) for 7 days after fasting from all food and drink (except water) for a minimum of 8 h prior to dosing. The dosing in the three MAD-cohorts was 100 mg, 200 mg and 300 mg of free anle138b equivalent, cohorts A_{M}, B_{M} and C_{M}, respectively. Post dosing mouth and hand checks were conducted to ensure the capsules were swallowed. In-study decisions were made by the safety advisory committee (SAC) comprising the principal investigator, the sponsor's medical monitor and a PK expert. For dose escalation to proceed, data needed to be available from a minimum of 6 subjects per cohort with completed per protocol safety and PK assessments up to 48 h after dosing to ensure at least 4 subjects had received active IMP. The decision to proceed to the next higher dose level was based on safety, tolerability and available PK data to 48 h post-administration. The following data were analyzed: Adverse events, vital signs, safety laboratory, ECG, physical examinations, plasma concentrations of anle138b with interim PK parameter estimations (Tmax, Cmax, AUC₀₋₂₄, AUC₀₋ₜₐᵤ, AUC₀₋ₗₐₛₜ and T½, where applicable). Data were provided to the SAC in accordance with CRO standard operating procedure (SOP) on interim dose decision-making and dose escalation.

In the food effect study (FES), the effect of food on the PK of anle138b was explored using a single dose of 150 mg anle138b, a dose level that was previously deemed safe and well tolerated in the SAD and MAD cohorts. This dose was administered either i) after a standard FDA-approved high-fat breakfast or ii) in the fasted state, i.e., fasted from all food and drink (except water) for a minimum of 8 h prior to dosing. In total, one cohort of 12 subjects was randomised in a 1:1 ratio to 2 treatment sequences (fed→fasted) or (fasted→fed). A minimum washout of at least 5 half-lives of anle138b between each dose was assured.

Outcome measures: The primary objective was to assess the safety and tolerability of single (SAD) and multiple (MAD) ascending doses of anle138b to healthy subjects in the fasted state and to assess the safety and tolerability of single doses of anle138b in both the fasted and fed state (FES). To this end, adverse events (AEs), clinical laboratory tests, vital signs, electrocardiograms (ECGs), QT interval corrected for heart rate using Fridericia's formula (QTcF), and physical examination findings were recorded. Participants were instructed to report all potential AEs immediately to the on-site personnel and were assessed by a physician before each dose of study drug. In addition, a physical examination of the relevant body system was performed in the event that a subject reports any new symptoms or AEs. AEs were defined following standard criteria, which are described in the protocol. The detailed PK parameters are provided in Tables 9-10, below. Any clinically significant abnormality in these assessments, including changes from baseline, were needed to be reported as an AE.

Secondary outcome measures were the oral PK of single (SAD) and multiple (MAD) ascending doses of anle138b in the fasted state as well as the effect of co-administration with food on the PK of anle138b. To this end PK blood samples were taken at day 1 (and for MAD day 7) pre-dose and at 0.5, 1, 1.5, 2, 3, 4, 6, 8, 10, 12, 16 and 20 h post-administration, at days 2 to 6 pre-dose only (MAD) and 24, 30, 36 and 48 h post final dose. Analysis of anle138b in plasma was done at Aptuit. Time point of last quantifiable data was seen to increase with an increase in dose as follows: SAD: from the 50 mg dose (subjects ranged between 8-24 h) to the 300 mg dose (subjects ranged between 36-48 h). MAD: from the 100 mg dose (subjects ranged between 8-36 h) to the 300 mg dose (subjects ranged between 20-48 h). In FES, subjects ranged between 24-48 h.

Data management was performed by the CRO using a validated electronic case report form (eCRF) database system and subjected to data consistency and validation checks. Data queries were raised within the study eCRF database by data management staff and resolved with the assistance of clinical staff.

AEs and medications were coded using the Medical Dictionary for Regulatory Activities (MedDRA) (v22.1). An independent coding review was performed within the Data Sciences department. Clinical chemistry and haematology data (and other safety laboratory data) were collected by a central laboratory (The Doctors Laboratory) and transferred electronically to the CRO. All demographic details and sample dates were cross-referenced with the corresponding data on the study database. Data was monitored by an external entity (Wirral Clinical Consultancy Ltd, Heswall, UK). Monitoring included the conduct of a site initiation visit, interim monitoring visits and a close-out visit. The database was closed after all queries had been resolved.

Statistical analysis: The study was exploratory and no formal sample size calculation was made. Based on experience from previous studies of similar design, eight subjects per cohort were enrolled for parts 1 (SAD) and 2 (MAD) and a total of twelve subjects for part 3 (FES). Populations and analysis sets were determined for safety and PK data after database lock using the criteria defined in the reporting and analysis plan. The safety population and safety analysis set for SAD and MAD were defined after database lock but prior to study unblinding. Statistical analysis and production of summary tables, figures and listings for all safety data (AEs, vital signs, ECGs and safety laboratory assessments) including changes from baseline as required in this study were performed using the statistical package SAS (v9.4). Additional statistics were provided for PK-related data including coefficient of variation (CV %), geometric mean, geometric CV % and geometric n (i.e., the number of subjects with an observation that were included in the natural logarithmic transformation). In SAD and MAD, dose proportionality was assessed. Formal statistical analysis was performed on the log-transformed PK parameters AUC₀₋ₗₐₛₜ, AUC_{0-inf}, and Cmax for SAD (day 1), and AUC₀₋ₜₐᵤ and Cmax for MAD (day 1 and day 7) to assess dose proportionality using the following power model:

$${log}_{e} ( AUC or Cmax ) = μ + β x {log}_{e} ( dose )$$

The relationship between the PK parameter (y) and dose is defined as follows:

$$y = α * dose β where y is AUC or Cmax .$$

Dose proportionality requires that β=1 for dose dependent parameters. This becomes a linear relationship after logarithmic transformation:

$$Log ( y ) = μ + β * log ( dose )$$

where μ=log (60 ) is the intercept and β is the slope from the linear model. Using this model it is possible to estimate β (measure of dose proportionality) and obtain 90% confidence interval (CI) for β (Gough, 1995; Smith, 2000). The estimate of β together with its 90% CI (βI, βu) was used to quantify the degree of non-proportionality. In MAD, formal statistical analysis was performed on the PK parameters AUC₀₋ₜₐᵤ and Cmax to assess dose accumulation. Log-transformed AUC₀₋ₜₐᵤ and Cmax was subjected to a mixed-effects model with treatment (dose level), day (day 1 or 7) and treatment-by-day interaction as fixed effects and subject as a random effect. The adjusted means obtained from the model, including differences for each comparison of interest and the associated 90% CIs, was back-transformed on the log scale to obtain adjusted geometric means, adjusted geometric mean ratios (GMRs) and 90% CIs of the ratio. The GMRs and 90% CIs were provided for each treatment and overall, i.e., day 7/day 1. In the FES, formal statistical analysis was performed on the PK parameters Cₘₐₓ, AUC₀₋ₗₐₛₜ and AUC₀₋₂₄ to assess the effects of food on anle138b. The PK parameters underwent a natural logarithmic transformation and were analysed using a mixed-effect model with terms for treatment (i.e., prandial state), period and sequence as fixed effects and subject nested within sequence as a random effect. Adjusted GMRs and 90% CIs for the adjusted GMRs for the comparison between fed and fasted states were provided where the ratios are defined as fed/fasted.

Results Of 196 individuals assessed for eligibility, 89 failed screening, 39 served as reserve subjects and 68 were included in the study. Of the included participants, 32 subjects (4 dosing groups of 8) were included in the SAD part, 24 subjects (3 dosing groups of 8) in the MAD part and 12 subjects in the FES. In the SAD part, 8 participants received placebo. In the MAD part, 6 participants received placebo. All participants completed the study as planned per protocol. There were no dropouts or early discontinuations. Demographic characteristics of the study population at baseline were similar for all cohorts and groups. Of 32 participants in the SAD part, 3 (9%) were female, all other participants including those in the MAD part and the FES were male. 100% of the study medication was taken as scheduled in all groups.

The primary read-outs of this trial were safety and tolerability. There were no serious adverse events (SAEs) and no AEs leading to study medication withdrawal in any part of the study. Treatment-emergent AEs were reported in comparable numbers in treated and placebo groups. There was no dose dependency with regard to AE reporting. All AEs completely recovered. In summary, daily oral administration of anle138b was shown to be safe and well tolerated up to the highest, daily dose of 300 mg per subject administered for up to 7 consecutive days. The secondary read-out was PK.

### Study anle138b-P1-01 Part 1 (SAD)

Table 9 shows Geometric Mean (CV %) key pharmacokinetic parameters of anle138b in healthy volunteers following single dose oral administration of anle138b DP. For Tmax Median (range) is shown. Abbreviations: Tₘₐₓ: Time to maximum peak; Cₘₐₓ: Maximum concentration; AUC: Area under the curve (=exposure); T_{1/2}: Plasma half-life; h: hour; ng: nanogram; ml: milliliter; NA: Not applicable. Maximum concentrations were achieved at between 0.5 and 2 hours post-administration (median Tmax of 1 to 1.5 hours post-administration).

TABLE 9
Geometric Mean (CV %) key pharmacokinetic parameters
of anle138b in healthy volunteers, fasted, following
single oral administration of anle138b DP

| | Tₘₐₓ* | Cₘₐₓ | AUC₍₀₋₂₄₎ | AUC₍₀₋ₗₐₛₜ₎ | T_{1/2} |
|---|---|---|---|---|---|
| Treatment | (h) | (ng/mL) | (ng · h/mL) | (ng · h/mL) | (h) |
| Cohort A | 1.00 | 54.3 | 113 | 109 | 3.942 |
| 50 mg | (0.50-2.00) | (58.5) | (66.0) | (68.4) | (201.3) |
| Cohort B | 1.50 | 156 | 366 | 372 | 10.799 |
| 100 mg | (0.50-2.00) | (30.5) | (23.4) | (26.6) | (57.6) |
| Cohort C | 1.50 | 458 | 1090 | 1130 | 12.761 |
| 200 mg | (1.00-2.00) | (56.3) | (55.8) | (57.2) | (30.9) |
| Cohort D | 1.50 | 704 | 1650 | 1720 | 16.221 |
| 300 mg | (1.50-2.00) | (49.5) | (48.6) | (47.1) | (32.3) |

*Median (range)

Overall, PK data showed drug was systemically available following oral administration with rapid absorption and a bi-phasic elimination. The practical terminal elimination half-time was ˜12 h. Potential therapeutic exposure (based upon nonclinical in vivo models) was already achieved after single 100 mg doses of anle138b. With increased doses of 200 or 300 mg, therapeutic exposures were increased correspondingly, without any relevant safety concerns.

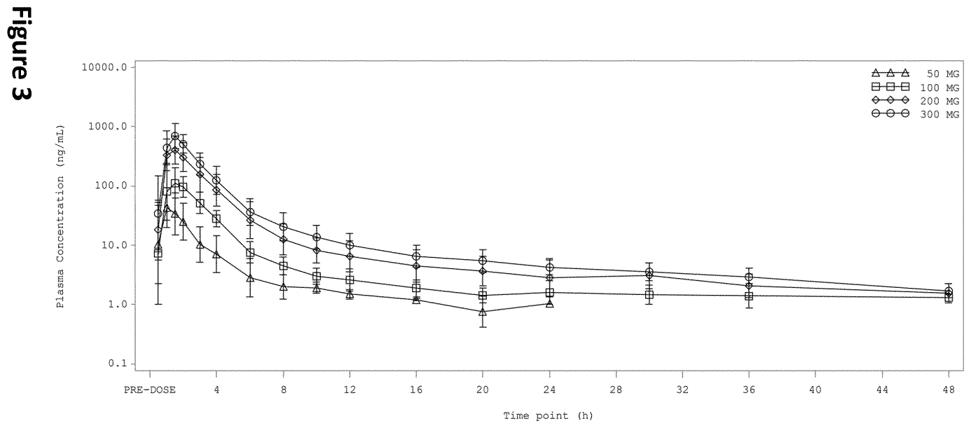

Following oral administration of the DP, plasma concentrations of anle138b became quantifiable at 0.5 hours post-administration in all subjects and remained quantifiable for up to 24 hours post-administration in the lowest dose group and for up to 48 hours post-administration in the highest dose group. Single Dose Plasma Concentrations of anle138b (FIG. 3, logarithmic plot).

Cmax values increased supra-proportionally over the 50 to 200 mg dose range and proportionally from 200 to 300 mg. A 2-fold increase in dose from 50 mg to 100 mg resulted in an approximate 2.9-fold increase in Cmax, and 3.3-fold increase in AUC. Another 2-fold increase in dose from 100 mg to 200 mg resulted in an approximate 2.9-fold increase in Cmax, and 3.0-fold increase in AUC. A 1.5-fold increase in dose from 200 mg to 300 mg resulted in an approximate 1.5-fold increase in Cmax, and 1.5-fold increase in AUC.

The elimination half-life of anle138b was variable. T_{1/2} was 3.9 hours in the 50 mg cohort and 10.8 h and 12.8 h, respectively, in the 100 mg and 200 mg cohorts. T_{1/2} increased to 16.2 h in the 300 mg group. The variability in T_{1/2} is suspected to be due to differences in the time of last quantifiable concentrations between subjects, leading to an inaccurate characterisation of the true terminal elimination phase especially in the 50 mg cohort. The change with dose may also be attributed to drug's auto-inhibition of its metabolic pathways such as CYP1A2.

### Study anle138b-P1-01 Part 2 (MAD)

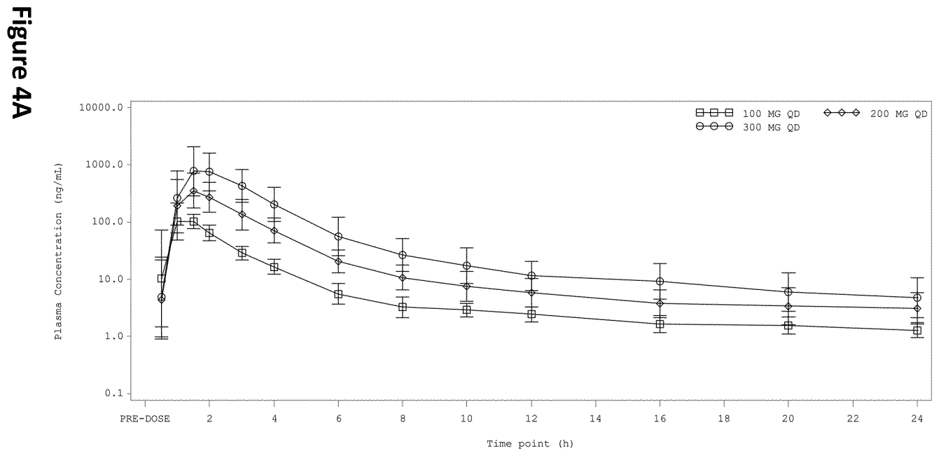

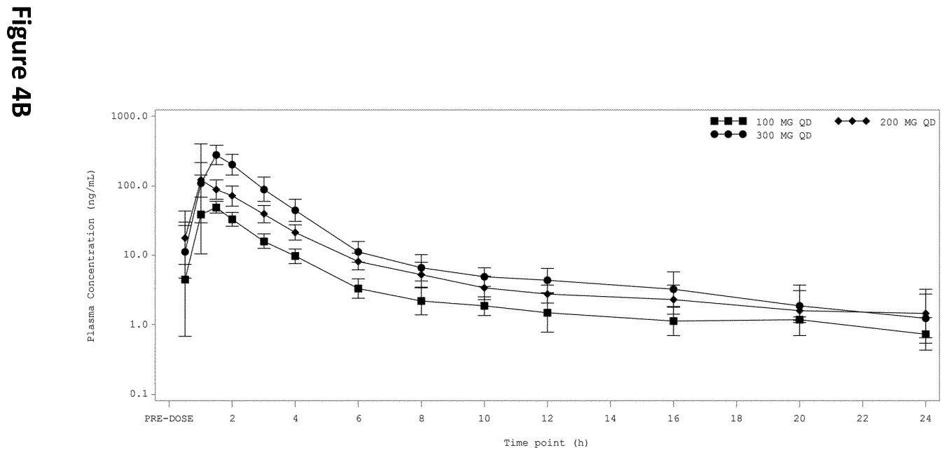

Following oral administration of anle138b in capsule form, plasma concentrations of anle138b became quantifiable at 0.5 hours post-administration in all subjects and remained quantifiable for up to 24 hours post-administration in all dose groups. This was seen both at Day 1 and at Day 7 (FIG. 4A and 4B, respectively).

Tₘₐₓ appeared to be relatively unaffected by multiple dosing. Maximum concentrations were achieved at between 1.0 and 2.0 hours post-administration (median Tₘₐₓ of 1 to 1.5 hours post-administration). On Day 1, Cₘₐₓ values increased supra-proportionally over the 100 to 300 mg dose range. A 2-fold increase in dose from 100 mg to 200 mg resulted in a 3.3-fold increase in Cₘₐₓ, and 3.5-fold increase in AUC. A 1.5-fold increase in dose from 200 mg to 300 mg resulted in a 2.0-fold increase in Cₘₐₓ, and 2.4-fold increase in AUC (Table 10). Repeated administration of anle138b capsules in the fasted state resulted in reductions in Cₘₐₓ and AUC exposures. Thus, compared to Day 1, Cₘₐₓ and AUC in the 100 mg group were reduced by about 53%, Cₘₐₓ and AUC in the 200 mg group were reduced by about 70%, and Cₘₐₓ and AUC in the 300 mg group were reduced by 66% and 71%. The accumulation ratios were therefore below 0.54 per cohort. The individual Cₘₐₓ and AUC values at Day 7 increased about dose-proportional with increasing doses.

Multiple dosing also tended to result in a decrease in half-life compared to the single ascending doses in Part 1 of the study, with the exception of the T_{1/2} in the 200 mg group that was about 10 hours and, thus, largely similar in MAD and in SAD. This presentation may relate to drug's auto-induction of its metabolic pathways.

TABLE 10
Geometric Mean (CV %) key pharmacokinetic parameters of
anle138b in healthy volunteers, fasted, following multiple
oral administration of anle138b in capsule form

| | | Tₘₐₓ* | Cₘₐₓ | AUC₍₀₋ₜₐᵤ₎ | T_{1/2} |
|---|---|---|---|---|---|
| Treatment | | (h) | (ng/mL) | (ng · h/mL) | (h) |
| Cohort A_{M} | Day 1 | 1.00 | 135 | 261 | NA |
| 100 mg | | (1.00-1.50) | (38.9) | (42.5) | |
| | Day 7 | 1.25 | 70.9 | 141 | 4.231 |
| | | (1.00-1.50) | (55.4) | (40.7) | (119.6) |
| Cohort B_{M} | Day 1 | 1.50 | 447 | 905 | NA |
| 200 mg | | (1.00-2.02) | (58.0) | (60.7) | |
| | Day 7 | 1.00 | 128 | 308 | 9.484 |
| | | (1.00-1.50) | (55.5) | (34.1) | (78.8) |
| Cohort C_{M} | Day 1 | 1.50 | 910 | 2210 | NA |
| 300 mg | | (1.50-2.00) | (94.9) | (81.1) | |
| | Day 7 | 1.50 | 307 | 633 | 6.068 |
| | | (1.00-1.50) | (55.9) | (37.3) | (0.1) |

*Median (range);
NA = not applicable

Overall, repeated daily dosing resulted in a ˜50 to ˜70% decrease in Cₘₐₓ and AUC of anle138b. These decreases with multiple dosing are in line with the results seen in repeat-dosing animal studies. The decreased exposure is likely a result of the induction of metabolising enzymes, such as CYP1A2 (oxidative metabolism). A reduction of levels of anle138b was also observed for trough (i.e., pre-dose) levels. Of note, steady state exposure appeared to be reached by day 5 based on trough levels.

In summary, the PK data showed that the study drug was safe and well tolerated at all dose levels. The practical elimination half-time was ˜12 h. Cₘₐₓ and AUC following single administrations of anle138b increased with dose. Potential therapeutic exposure levels (AUC₍₀₋₂₄₎>300 ng*h/ml) were obtained with the 100 mg dose. Maximum concentrations were achieved between 0.5 and 2 hours post-administration (median Tₘₐₓ of 1 to 1.5 hours post-administration). The terminal elimination half-time was ˜12 h. The MAD part of the study generally confirmed the Tₘₐₓ. Cₘₐₓ and AUC values following 7-day daily dosing were decreased by approximately 50% to ˜70%, likely due to induction of metabolising CYP enzymes, particularly CYP1A2.

Overall, potential therapeutic exposure based upon nonclinical in vivo models was achieved after a single 100 mg dose. With increased doses of 200 or 300 mg, exposure levels were increased correspondingly, without any safety concerns.

### Study Part 3: Food Effect

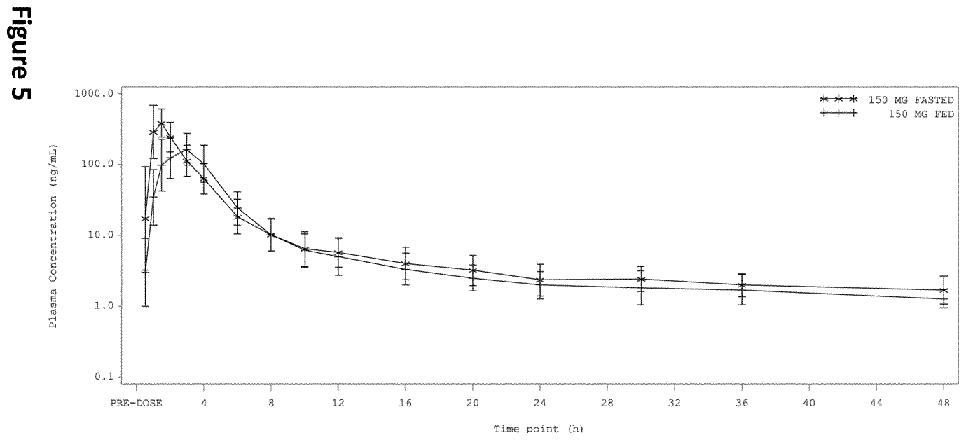

Following oral administration of anle138b under both fasted and fed conditions, plasma concentrations of anle138b became quantifiable between 0.5 and 1.0 hours post-administration and remained quantifiable for up to 24 to 48 hours post-administration (FIG. 5)

Maximum concentrations were achieved between 1 and 2 hours post-administration (median Tₘₐₓ of 1.25 hours post-administration) in the fasted state and between 1.5 and 3 hours post-administration (median Tₘₐₓ of 3.00 hours post-administration) in the fed state (Table 10). A change in prandial state from fasted to fed resulted in approximately 74% (90% CI: 61%, 84%) of the dose being bioavailable in the fed state compare to the fasted state. Cₘₐₓ was decreased by about half with food (mean Cmax fed 196 ng/ml (range 110-479), fasted 442 ng/ml (range 225-1420)), in line with the delay in Tₘₐₓ. Total exposure was less affected following administration with food (mean AUC₀₋₂₄ fasted 896 ng*h/ml (range 433-2720), fed 641 ng*h/mL (range 378-1920)). Inter-subject variability with relation to exposure (Cₘₐₓ and AUC) was moderate across both regimens. Overall, the range of exposure observed was overlapping for fed and fasted dosing.

The elimination half-life of anle138b was only slightly higher in the prandial state, with geometric mean values of 11.3 and 15.2 hours, following administration in the fasted and fed regimens respectively.

TABLE 11
Geometric Mean (CV %) key pharmacokinetic parameters
of anle138b in healthy volunteers following single oral
administration of anle138b in fasted and fed state

| | Tₘₐₓ* | Cₘₐₓ | AUC₍₀₋₂₄₎ | AUC₍₀₋ₗₐₛₜ₎ | T_{1/2} |
|---|---|---|---|---|---|
| Treatment | (h) | (ng/mL) | (ng · h/mL) | (ng · h/mL) | (h) |
| Cohort A | 1.25 | 442 | 896 | 888 | 11.341 |
| 150 mg | (1.00-2.00) | (58.7) | (54.7) | (55.8) | (17.4) |
| fasted | | | | | |
| Cohort B | 3.00 | 196 | 641 | 668 | 15.218 |
| 150 mg | (1.50-3.08) | (48.6) | (49.0) | (50.0) | (28.3) |
| fed | | | | | |

Overall, the PK data showed that the study drug was safe and well tolerated following both fasted and fed conditions. Treatment under fed conditions led to a delay in time to maximum concentration, which can be expected based on the altered kinetics of IMP uptake in a filled compared to an empty stomach. Cmax was decreased by about half with food, in line with the delay in Tₘₐₓ.

Administration of anle138b with food led to a slight decrease in total exposure, but the decrease is not considered clinically relevant for dosing, as a largely overlapping range of exposure levels was observed for fed and fasted dosing.

### Example 2.2 Phase 1 Clinical Study in PD Patients

Study Title: A Single Part Study to Assess the Safety, Tolerability, Pharmacokinetics and Pharmacodynamics of Multiple Ascending Oral Doses of anle138b, and to Characterize the Effect of Food on the Pharmacokinetics of anle138b in Patients with Mild to Moderate Parkinson's Disease.

This was a two-center, double-blind, randomised, placebo-controlled study that enrolled twenty two (22) participants in cohorts (i.e. regimens) A-C. All completed the study per protocol. After completion of Cohorts A to C it was decided to add Cohorts D and E of 24 subjects. Cohort D expanded sample size of previous dosing regiments 150 mg and 300 mg QD and Cohort E administration of anle138b 300 mg for 28 days, under non fasted conditions.

Drug product used is described in section 1.3, supra, placebo was vehicle in capsule. Subjects were between 50 and 80 years of age with a diagnosis of idiopathic PD as defined by the Movement Disorders Society criteria (either fulfilling criteria for “Clinically Established PD” or for “Clinically Probable PD”). They had to present with Hoehn and Yahr stage I-III (i.e. able to walk unaided), and stable medication for PD for 1 month prior to inclusion and anticipated over the study period. The subjects were randomly assigned to receive one of three drug regimens, A, B or C, as follows:
Regimen A—150 mg (5×30 mg) anle138b oral capsules or matching placebo QD, for 7 consecutive days in the fasted state, followed by a single administration of 150 mg anle138b oral capsule or matching placebo on Day 9 in the fed state,
Regimen B—300 mg (10×30 mg) anle138b oral capsules or matching placebo QD, for 7 consecutive days in the fasted state, followed by a single administration of 300 mg anle138b oral capsule (10×30 mg capsules) or matching placebo on Day 9 in the fed state,
Regimen C—150 mg (5×30 mg) anle138b oral capsules or matching placebo BID (total 300 mg daily), for 7 consecutive days in the fasted state.

Subjects in Regimen E were randomly assigned to receive QD doses of either 300 mg (10×30 mg) anle138b oral capsules or matching placebo, for 28 consecutive days in the non fasted state.

Fed state conditions were considered moderate with standard size breakfast; 570 kcal including 24% fat.

Primary endpoints provided safety and tolerability information for the test product by assessing: AEs, vital signs, ECGs, physical examinations and laboratory safety tests, and to provide additional safety and tolerability information for the test product taken also in the fed state by assessing: AEs, vital signs, ECGs, physical examinations and laboratory safety tests.

Secondary endpoints provided PK information for the test product in PD patients by assessing plasma exposure under fast and fed conditions.

Pharmacodynamic assessments included Movement Disorder Society-sponsored revision of the Unified PD Rating Scale (MDS-UPDRS). An interim data review following each cohort was performed to agree progression and the dose level for the next cohort.

To allow for an additional safety factor of 2 compared with the highest dose tested in the first in human (FIH) study (i.e. 300 mg), a dose of 150 mg of anle138b was selected for the first dosing cohort of the study (see example 2.1, above).

Results: data show that doses up to 300 mg of anle138b were safe and well tolerated under the tested conditions. Furthermore, PK data were similar to the PK data obtained in the FIH, healthy volunteer study. Table 12 provides the geometric mean (CV %) key pharmacokinetic parameters of anle138b in patients on Day 1 and Day 7 in the fasted state following multiple oral administration of anle138b in capsule form.

The 28 day dosing of 300 mg of anle138b has comparable PK trends as observed following 7 day dosing. Concentrations are expected to be in similar ranges. Therefore, efficacy response is expected not be jeopardized, with general well tolerated safety profile.

TABLE 12
Geometric mean (CV %) key PK parameters of anle138b in patients on Day 1 and Day 7 in
the fasted state following multiple oral administration of anle138b in capsule form.

| | | Tmax | Cmax | Ctau ^{ab} | AUC(0-tau) ^{ab} | T_{1/2} | AR | AR |
|---|---|---|---|---|---|---|---|---|
| | Day | (h) | (ng/mL) | (ng/mL) | (ng · h/mL) | (h) | Cmax | AUC ^{ab} |
| Regimen A | 1 | 1.25 | 462 | 3.76 | 1040 | N/A | N/A | N/A |
| [n = 6 active] | | (1.00-1.50) | (66.4) | (44.3) | (54.6) | | | |
| 150 mg, | 7 | 1.00 | 160 | 1.86 | 388 | 16.25, | 0.346 | 0.371 |
| fed, QD | | (1.00-2.00) | (43.5) | (53.0) | (40.7) | 17.10^{c} | (62.8) | (39.8) |
| | | | | | | [n = 2] | | |
| Regimen B | 1 | 1.50 | 933 | 6.81 | 2080 | N/A | N/A | N/A |
| [n = 6 active] | | (1.00-4.00) | (70.3) | (29.6%) | (29.4%) | | | |
| 300 mg, | | | | [n = 5] | [n = 5] | | | |
| fed, QD | 7 | 1.6 | 323 | 3.19 | 729 | 11.815 | 0.428 | 0.351 |
| | | (1.00-2.00) | (46.2%) | (21.3%) | (22.9%) | (26.6%) | (80.7) | (50.7%) |
| | | [n = 5] | [n = 5] | [n = 5] | [n = 5] | [n = 4] | [n = 5] | [n = 5] |
| Regimen C | 1 | 1.00 | 546 | 7.41 | 993 | N/A | N/A | N/A |
| [n = 5 active] | | (1.00-2.00) | (47.5) | (88.6%) | (56.0) | | | |
| 150 mg, | | | | | | | | |
| fast, BID | 7 | 1.00 | 182 | 4.05 | 297 | 14.1 | 0.333 | 0.299 |
| | | (1.00-1.50) | (56.0) | (55.2) | (31.7) | (45.7) | (53.9) | (37.4) |

*median (range),
^{a} based on a tau of 24 h when dosing QD,
^{b} based on tau of 12 h when dosing BID,
N/A—Not Applicable;
^{c}Individual subject values

Table 13 shows the geometric mean (CV %) key pharmacokinetic parameters of anle138b in patients following a single oral administration of anle138b in capsule form on Day 9 in the fed state.

TABLE 13
Geometric mean (CV %) key PK parameters of anle138b in patients following a single
oral administration of anle138b in capsule form on Day 9 in the fed state

| | | | | | | | | ^{a}Frel | ^{a}Frel |
|---|---|---|---|---|---|---|---|---|---|
| | Tmax* | Cmax | C24 | AUC(0-24) | AUC(0-last) | AUC(0-inf) | T_{1/2} | Cmax | AUC(0-24) |
| | (h) | (ng/mL) | (ng/mL) | (ng · h/mL) | (ng · h/mL) | (ng · h/mL | (h) | (%) | (%) |
| Regimen A | 3.00 | 167 | 1.87 | 513 | 542 | 599 | 15.9 | 104 | 132 |
| 150 mg | (1.50-4.00) | (43.2) | (41.6) | (30.9) | (31.8) | (15.7%) | (26.8%) | (38.9) | (16.8) |
| [n = 6 active] | | | | | | [n = 3] | [n = 3] | | |
| Regimen B | 3.00 | 231 | 2.88 | 795 | 831 | 826 | 13.0 | 71.4 | 109 |
| 300 mg | (3.00-4.00) | (26.1%) | (40.5%) | (30.3%) | (30.9%) | (33.1%) | (14.4%) | (34.3) | (12.9) |
| [n = 6 active] | [n = 5] | [n = 5] | [n = 5] | [n = 5] | [n = 5] | [n = 4] | [n = 4] | [n = 5] | [n = 5] |

*median (range)
^{a}Frel: Comparison Day 9 fed vs Day 7 QD fasted.
Note:
Day 9 fed dosing was only investigated in Regimens A and B

Following single oral administration of anle138b in capsule form at 150 mg (Regimens A [QD] and C [BID]) and 300 mg (Regimen B (QD)) on Day 1 in the fasted state, peak plasma concentrations (Cmax) of anle138b occurred between 1.00-1.50 hours (h) post-administration, 1.00-4.00 h post-administration and 1.00-2.00 h post-administration, respectively, and median Tmax occurred at 1.25 h, 1.50 h and 1.00 h post-administration respectively for Regimens A, B and C.

Following once daily oral administration of anle138b in capsule form at 150 mg (Regimens A [QD] and C [BID]) and 300 mg (Regimen B [QD]) for 7 days, peak plasma concentrations of anle138b occurred between 1.00-2.00 h post-administration, 1.00-2.00 h post-administration and between 1.00-1.50 h post-administration, respectively, and median Tmax occurred at 1.00 h, 1.50 h and 1.00 h post-administration, respectively, for Regimens A, B and C.

Following once and twice daily oral administration for 7 days, systemic exposure appeared to decrease in all three regimens, with geometric mean accumulation ratios on Day 7 of 0.346, 0.428 and 0.333 for Cmax and values of 0.371, 0.351 and 0.299 for AUC(0-tau) respectively for Regimens A, B and C. The geometric mean Ty values were 11.8 h and 14.1 h, respectively for Regimens B and C. The individual terminal half-lives following administration of 150 mg anle138b QD for 7 days (Regimen A) were 16.25 h and 17.10 h.

Following single oral administration of DP at 150 mg and 300 mg in the fed state on Day 9, peak plasma concentrations (Cmax) of anle138b occurred between 1.50-4.00 h post dose and between 3.00 h-4.00 h post-administration, respectively. Median Tmax occurred at 3.00 h post-administration following dosing of both regimens. The geometric mean terminal T_{½} were 15.9 h and 13.0 h, respectively at the 150 mg and 300 mg dose levels.

The geometric mean relative bioavailability of anle138b when administered in the presence of food following a single dose on Day 9, relative to when fasted on Day 7 was 104% and 71.4% based on Cmax and 132% and 109% based on AUC(0-24), respectively at the 150 mg and 300 mg dose levels.

Following single oral administration of anle138b in capsule form at 150 mg (Regimens A-QD and C-BID) and 300 mg (Regimen B-QD) on Day 1 in the fasted state, the maximum individual Cmax and AUC (0-24) values were approximately 24.9% and 6.19%, 75.8% and 8.4% and 26.4% and 4.94% of the exposure limits of anle138b outlined in the protocol, respectively for Regimens A, B and C. (Note that for Regimen C, AUC(0-tau) on this dosing occasion was equivalent to AUC(0-12), with exposures limits based on AUC(0-24)).

Following multiple QD oral doses of 300 mg anle138b to male and female patients with mild to moderate PD in the fasted state, the geometric mean (geometric CV %) CSF anle138b concentration was 0.3186 ng/ml (108.1%) and the geometric mean (geometric CV %) plasma anle138b concentration was 132.593 ng/ml (58.5%) at 3 h post-dose on Day 5. The geometric mean (geometric CV %) CSF/plasma anle138b concentration ratio was 0.00240 (45.0%).

### REFERENCES

Berg D, et al. Movement disorder society criteria for clinically established early Parkinson's disease. Mov Disord 2018; 33(10):1643-6.
Brendel M, et al. Late-stage Anle138b treatment ameliorates tau pathology and metabolic decline in a mouse model of human Alzheimer's disease tau. Alzheimers Res Ther 2019; 11(1):67.
Cacabelos R., Parkinson's Disease: From Pathogenesis to Pharmacogenomics. Int J Mol Sci. 2017, 18(3):551
Camilleri A, et al. Tau-induced mitochondrial membrane perturbation is dependent upon cardiolipin. Biochim Biophys Acta Biomembr 2020; 1862(2):183064.
Deeg A A, et al. Anle138b and related compounds are aggregation specific fluorescence markers and reveal high affinity binding to alpha-synuclein aggregates. Biochim Biophys Acta 2015; 1850(9):1884-90.
Ghio S, et al. Cardiolipin Promotes Pore-Forming Activity of Alpha-Synuclein Oligomers in Mitochondrial Membranes. ACS Chem Neurosci 2019; 10(8):3815-29.
Gilman S, et al. Second consensus statement on the diagnosis of multiple system atrophy. Neurology 2008; 71(9):670-6.
Gough et al., “Assessment of Dose Proportionality: Report from the Statisticians in the Pharmaceutical Industry/Pharmacokinetics UK Joint Working Party”, Drug Information Journal, 1995, 29:1039-1048.
Heras-Garvin A, et al. Anle138b modulates alpha-synuclein oligomerization and prevents motor decline and neurodegeneration in a mouse model of multiple system atrophy. Mov Disord 2019; 34(2):255-63.
Jannin, V. Lauroyl polyoxylglycerides, functionalized coconut oil, enhancing the bioavailability of poorly soluble active substances. OCL J. 2009. 16:4, 267-272.
Levin J, et al. The oligomer modulator anle138b inhibits disease progression in a Parkinson mouse model even with treatment started after disease onset. Acta Neuropathol 2014; 127(5):779-80.
Levin J, et al. The Differential Diagnosis and Treatment of Atypical Parkinsonism. Dtsch Arztebl Int 2016; 113(5):61-9.
Levin J, et al. Safety, tolerability and pharmacokinetics of the oligomer modulator anle138b with exposure levels sufficient for therapeutic efficacy in a murine Parkinson model: A randomised, double-blind, placebo-controlled phase 1a trial. EBioMedicine. 2022; 80:104021; doi: 10.1016/j.ebiom.2022.104021.
Martinez Hernandez A, et al. The diphenylpyrazole compound anle138b blocks Abeta channels and rescues disease phenotypes in a mouse model for amyloid pathology. EMBO Mol Med 2018; 10(1):32-47.
Matthes D, et al. Resolving the Atomistic Modes of Anle138b Inhibitory Action on Peptide Oligomer Formation. ACS Chem Neurosci 2017; 8(12):2791-808.
McKeith I G, et al. Diagnosis and management of dementia with Lewy bodies: Fourth consensus report of the DLB Consortium. Neurology 2017; 89(1):88-100.
Meissner W G, et al. Multiple System Atrophy: Recent Developments and Future Perspectives. Mov Disord 2019; 34(11):1629-42.
Panigrahi K C et al. Gelucire: a versatile polymer for modified release drug delivery system. Future J Pharma Sci 2018. 4:102-108.
Reiner A M, et al. Photophysics of diphenyl-pyrazole compounds in solutions and a-synuclein aggregates. Biochim Biophys Acta Gen Subj 2018; 1862(4):800-7.
Smith, B. et al., “Confidence Interval Criteria for Assessment of Dose Proportionality”, Pharmaceutical Research, 2000 17(10):1278-1283.
Strickley, R G. Solubilizing Excipients in Oral and Injectable Formulations. Pharma Res, 2004. 21(2):201-230.
Wagner J, et al. Reducing tau aggregates with anle138b delays disease progression in a mouse model of tauopathies. Acta Neuropathol 2015; 130(5):619-31.
Wagner J, et al. Anle138b: a novel oligomer modulator for disease-modifying therapy of neurodegenerative diseases such as prion and Parkinson's disease. Acta Neuropathol 2013; 125(6):795-813.
Wegrzynowicz M, et al. Depopulation of dense alpha-synuclein aggregates is associated with rescue of dopamine neuron dysfunction and death in a new Parkinson's disease model. Acta Neuropathol 2019; 138(4):575-95.
Wong Y C, and D. Krainc. alpha-Synuclein toxicity in neurodegeneration: mechanism and therapeutic strategies. Nat Med 2017; 23(2):1-13.

All patent documents and publications disclosed herein are herein incorporated by reference to the same extent as if each individual publication was specifically and individually incorporated by reference.

The invention illustratively described herein suitably may be practiced in the absence of any element(s) not specifically disclosed herein. Thus, for example, in each instance herein any of the terms “comprising”, and “consisting of” may be replaced with either of the other two terms.

For the foregoing embodiments, each embodiment disclosed herein is contemplated as being applicable to each of the other disclosed embodiments. For example, the elements recited in the composition embodiments can be used in the use embodiments described herein and vice versa. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be contemplated by those skilled in the art, and that such modifications and variations are considered to be within the scope of this invention as defined by the appended claims.

1. A pharmaceutical composition comprising:
at least one compound having the general formula (A) or (A*) 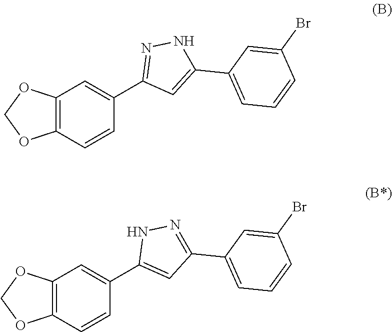
or a stereoisomer, racemate, hydrate or solvate thereof,
wherein
R is selected from hydrogen; C₁₋₄ alkyl; and —C₁₋₄ alkylene-halogen;
Hal is selected from F, Cl, Br, and I; and
R^{E7} and R^{E8} are independently H or F;
and a pharmaceutically acceptable excipient, wherein the excipient comprises at least one monoester of a fatty acid and polyethylene glycol and/or at least one diester of a fatty acid and polyethylene glycol,
wherein
the fatty acid is independently selected from C₈-C₂₂ fatty acids; and
the polyethylene glycol is independently selected from polyethylene glycols containing about 20 to about 40 ethylene oxide units.

2. The pharmaceutical composition of claim 1, wherein the pharmaceutical composition comprises a compound having the general formula (B), (B*) 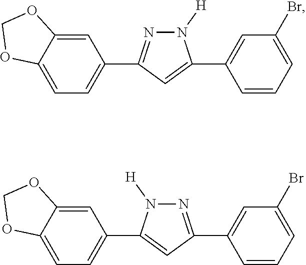
or a mixture thereof.

3. The pharmaceutical composition of claim 1 wherein the excipient further comprises a monoglyceride of a fatty acid, a diglyceride of a fatty acid and/or a triglyceride of a fatty acid, wherein the fatty acid is independently selected from C₈-C₂₂ fatty acids, preferably C₈-C₁₈ fatty acids.

4. The pharmaceutical composition of claim 1, wherein the polyethylene glycol contains about 32 ethylene oxide units.

5. The pharmaceutical composition of claim 1, wherein the excipient further comprises a polyethylene glycol containing about 20 to about 40 ethylene oxide units, preferably about 32 ethylene oxide units.

6. The pharmaceutical composition of claim 1, wherein the fatty acid comprises lauric acid, preferably wherein the fatty acid comprises 30 to 50 wt % lauric acid based on the total weight of fatty acids.

7. The pharmaceutical composition of claim 1, wherein the excipient comprises a mixture of monoesters of fatty acids and polyethylene glycol and/or diesters of fatty acids and polyethylene glycol, wherein the fatty acids are derived from coconut oil and/or hydrogenated coconut oil.

8. The pharmaceutical composition of claim 1, wherein the excipient comprises a mixture of monoesters of fatty acids and polyethylene glycol and/or diesters of fatty acids and polyethylene glycol, wherein the fatty acids comprise
up to 15 wt % caprylic acid (C8),
up to 12 wt % capric acid (C10),
30 to 50 wt % lauric acid (C12),
5 to 25 wt % myristic acid (C14),
4 to 25 wt % palmitic acid (C16), and
5 to 35 wt % stearic acid (C18).

9. The pharmaceutical composition of claim 1, wherein the excipient comprises about 50 wt % to about 80 wt %, preferably about 60 wt % to about 75 wt %, more preferably about 72 wt %, of the at least one monoester of a fatty acid and polyethylene glycol and/or the at least one diester of a fatty acid and polyethylene glycol.

10. The pharmaceutical composition of claim 3, wherein the excipient comprises about 10 wt % to about 30 wt %, preferably about 15 wt % to about 25 wt %, more preferably about 20 wt %, of the monoglyceride of the fatty acid, the diglyceride of the fatty acid and/or the triglyceride of the fatty acid.

11. The pharmaceutical composition of claim 5, wherein the excipient comprises about 5 wt % to about 20 wt %, preferably about 5 wt % to about 10 wt %, more preferably about 8 wt %, of the polyethylene glycol containing about 20 to about 40 ethylene oxide units.

12. The pharmaceutical composition of claim 1, wherein the excipient is obtained by an alcoholysis reaction between the polyethylene glycol and a triglyceride of the fatty acid.

13. The pharmaceutical composition of claim 1, wherein the excipient has a melting range in the range of about 33° C. to about 64° C., preferably about 35° C. to about 55° C., more preferably about 42.5° C. to about 47.5° C., even more preferably about 44° C.

14. The pharmaceutical composition of claim 1, wherein the excipient has a hydrophilic lipophilic balance (HLB) of about 1 to about 16, preferably from about 7 to about 14, about 11 or about 14.

15. The pharmaceutical composition of claim 1, wherein the pharmaceutical composition comprises about 3 wt % to about 5 wt % of the compound having the general formula (A) or (A*) and about 95 wt % to about 97 wt % of the excipient, based on 100 wt % of the total pharmaceutical composition.

16. An oral dosage form comprising the pharmaceutical composition of claim 1, the dosage form comprising about 1 mg to about 100 mg of the compound, or about 5 mg to about 50 mg of the compound, preferably about 10 mg or about 30 mg of the compound.

17. The oral dosage form of claim 16, in the form of a capsule.

18. A method of treating or preventing a disease linked to protein aggregation and/or a neurodegenerative disease, wherein a therapeutically effective amount of a pharmaceutical composition of claim 1 is administered to a subject in need thereof.

19. The method of claim 18, wherein the disease is an α-synucleinopathy.

20. The method of claim 19, wherein the synucleinopathy is multiple system atrophy (MSA), Parkinson's disease (PD), or dementia with Lewy bodies (DLB), preferably multiple system atrophy (MSA).

21. The method of claim 18, wherein the pharmaceutical composition is administered orally and is administered to a subject without regard to food intake.
